(19) United States
(12) Patent Application Publication
(10) Pub. No.: US 2023/0101523 A1
(43) Pub. Date: Mar. 30, 2023

(54) END-TO-END APTAMER DEVELOPMENT SYSTEM

(71) Applicant: X Development LLC, Mountain View, CA (US)

(72) Inventors: Ryan Poplin, Newark, CA (US); Lance Co Ting Keh, La Crescenta, CA (US); Ivan Grubisic, Oakland, CA (US); Ray Nagatani, San Francisco, CA (US)

(73) Assignee: X Development LLC, Mountain View, CA (US)

(21) Appl. No.: 17/936,181

(22) Filed: Sep. 28, 2022

Related U.S. Application Data
(60) Provisional application No. 63/249,709, filed on Sep. 29, 2021.

(51) Int. Cl. G16B 30/10; C12N 15/115; G06N 3/08; G16B 35/20; G06N 20/20; G16B 40/20

(52) U.S. Cl. CPC G16B 30/10; C12N 15/115; G06N 3/08; G16B 35/20; G06N 20/20; G16B 40/20

(57) ABSTRACT

The present disclosure relates to in vitro experiments and in silico computation and machine-learning based techniques to iteratively improve a process for identifying binders that can bind a target. Particularly, aspects of the present disclosure are directed to obtaining initial sequence data, identifying, by a first machine-learning model having model parameters learned from the initial sequence data, a first set of aptamer sequences, obtaining, using an in vitro binding selection process, subsequent sequence data including sequences from the first set of aptamer sequences, identifying, by a second machine-learning model having model parameters learned from the subsequent sequence data, a second set of aptamer sequences, determining, using one or more in vitro assays, analytical data for aptamers synthesized from the second set of aptamer sequences, and identifying a final set of aptamer sequences from the second set of aptamer sequences based on the analytical data associated with each aptamer.

### CROSS-REFERENCE TO RELATED APPLICATION

This application claims the benefit of and the priority to U.S. Provisional Application No. 63/249,709, filed on Sep. 29, 2021, which is hereby incorporated by reference in its entirety for all purposes.

### FIELD

The present disclosure relates to development of aptamers, and in particular to a closed loop aptamer development system that leverages in vitro experiments and in silico computation and machine-learning based techniques to iteratively improve a process for identifying binders that can bind a molecular target.

### BACKGROUND

Aptamers are short sequences of single-stranded oligonucleotides (e.g., anything that is characterized as a nucleic acid, including xenobases). The sugar backbone of the single-stranded oligonucleotides functions as the acid and the A (adenine), T (thymine), C (cytosine), G (guanine) refers to the base. An aptamer can involve modifications to either the acid or the base. Aptamers have been shown to selectively bind to specific targets (e.g., proteins, protein complexes, peptides, carbohydrates, inorganic molecules, organic molecules such as metabolites, cells, etc.) with high binding affinity. Further, aptamers can be highly specific, in that a given aptamer may exhibit high binding affinity for one target but low binding affinity for many other targets. Thus, aptamers can be used to (for example) bind to disease-signature targets to facilitate a diagnostic process, bind to a treatment target to effectively deliver a treatment (e.g., a therapeutic or a cytotoxic agent linked to the aptamer), bind to target molecules within a mixture to facilitate purification, bind to a target to neutralize its biological effects, etc. However, the utility of an aptamer hinges on a degree to which it effectively binds to a target.

Frequently, an iterative experimental process (e.g., Systematic Evolution of Ligands by EXponential Enrichment (SELEX)) is used to identify aptamers that are selectively bound to target molecules with high affinity. In the iterative experimental process, a nucleic acid library of oligonucleotide strands (aptamers) is incubated with a target molecule. Then, the target-bound oligonucleotide strands are separated from the unbound strands and amplified via polymerase chain reaction (PCR) to seed a new pool of oligonucleotide strands. This selection process is continued for a number (e.g., 6-15) rounds with increasingly stringent conditions, which ensure that the oligonucleotide strands obtained have the highest affinity to the target molecule.

The nucleic acid library typically includes 10¹⁴-10¹⁵ random oligonucleotide strands (aptamers). However, there are approximately a septillion (10²⁴) different aptamers that could be considered. Exploring this full space of candidate aptamers is impractical. However, given that present-day experiments are now only a sliver of the full space, it is highly likely that optimal aptamer selection is not currently being achieved. This is particularly true when it is important to assess the degree to which aptamers bind with multiple different targets, as only a small portion of aptamers will have the desired combination of binding affinities across the targets. Accordingly, while substantive studies on aptamers have progressed since the introduction of the SELEX process, it would take an enormous amount of resources and time to experimentally evaluate a septillion (10²⁴) different aptamers every time a new target is proposed. In particular, there is a need for improving upon current experimental limitations with scalable machine-learning modeling techniques to identify aptamers and derivatives thereof that selectively bind to target molecules with high affinity.

### SUMMARY

In various embodiments, a method is provided that comprises: (a) obtaining initial sequence data for aptamers of an initial aptamer library that bind to a target, do not bind to the target, or a combination thereof; (b) identifying, by a first machine-learning model, a first set of aptamer sequences as satisfying one or more constraints, where the first machine-learning model comprises model parameters learned from the initial sequence data, and the first set of aptamer sequences are derived from a subset of sequences from the initial sequence data, sequences from a pool of sequences different from sequences from the initial sequence data, or a combination thereof; (c) obtaining, using an in vitro binding selection process, subsequent sequence data for aptamers of a subsequent aptamer library that bind to the target, do not bind to the target, or a combination thereof, where the subsequent aptamer library comprises aptamers synthesized from the first set of aptamer sequences; (d) identifying, by a second machine-learning model, a second set of aptamer sequences as satisfying the one or more constraints, where the second machine-learning model comprises model parameters learned from the subsequent sequence data, and the second set of aptamer sequences are derived from a subset of sequences from the subsequent sequence data, sequences from a pool of sequences different from sequences from the subsequent sequence data, or a combination thereof; (e) determining, using one or more in vitro assays, analytical data for aptamers synthesized from the second set of aptamer sequences; (f) identifying a final set of aptamer sequences from the second set of aptamer sequences that satisfy the one or more constraints based on the analytical data associated with each aptamer; and (g) outputting the final set of aptamer sequences.

In some embodiments, the method further comprises training the first machine-learning algorithm using the initial sequence data to learn the model parameters and generate the first machine-learning model, where the initial sequence data comprises aptamer sequences and associated analytical data, the analytical data comprising a first binding-approximation metric, a first functional-approximation metric, or a combination thereof of aptamers derived from the aptamer sequences.

In some embodiments, the method further comprises training the second machine-learning algorithm using the subsequent sequence data to learn the model parameters and generate the second machine-learning model, where the subsequent sequence data comprises aptamer sequences and associated analytical data, the analytical data comprising a second binding-approximation metric, a second functional-approximation metric, or a combination thereof of aptamers derived from the aptamer sequences.

In some embodiments, prior to identifying the second set of aptamer sequences, the first machine-learning algorithm is retrained using the subsequent sequence data to relearn the model parameters and generate another version of the first machine-learning model, where the subsequent sequence data comprises aptamer sequences and associated analytical data, the analytical data comprising a second binding-approximation metric, a second functional-approximation metric, or a combination thereof of aptamers derived from the aptamer sequences; step (b) is repeated using the another version of the first machine-learning model to identify a revised first set of aptamer sequences as satisfying the one or more constraints; and step (c) is repeated to identify subsequent sequence data where the subsequent aptamer library comprises aptamers synthesized from the revised first set of aptamer sequences.

In some embodiments, prior to identifying the final set of aptamers, the first machine-learning algorithm is retrained using the second set of aptamer sequences and the analytical data for aptamers derived from the second set of aptamer sequences to relearn the model parameters and generate another version of the first machine-learning model, where the analytical data comprises a third binding-approximation metric, a third functional-approximation metric, or a combination thereof of aptamers derived from the second set of aptamer sequences; step (b) is then repeated using the another version of the first machine-learning model to identify a revised first set of aptamer sequences as satisfying the one or more constraints; next, step (c) is repeated to obtain revised subsequent sequence data, where the subsequent aptamer library comprises aptamers synthesized from the revised first set of aptamer sequences; and steps (d)-(e) are repeated based on the revised subsequent sequence data.

In some embodiments, prior to identifying the final set of aptamers, the second machine-learning algorithm is retrained using the second set of aptamer sequences and the analytical data for aptamers derived from the second set of aptamer sequences to relearn the model parameters and generate another version of the second machine-learning model, where the analytical data comprises a third binding-approximation metric, a third functional-approximation metric, or a combination thereof of aptamers derived from the second set of aptamer sequences; then step (d) is repeated using the another version of the second machine-learning model to identify a revised second set of aptamer sequences as satisfying the one or more constraints; and step (e) is repeated to determine analytical data for aptamers derived from the revised second set of aptamer sequences.

In some embodiments, the method further comprises: synthesizing one or more aptamers using the final set of aptamer sequences; and synthesizing a biologic using the one or more aptamers.

In some embodiments, a system is provided that includes one or more data processors and a non-transitory computer readable storage medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable storage medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure will be better understood in view of the following non-limiting figures, in which:

FIG. 1 shows a block diagram of a pipeline for strategically identifying and generating high affinity binders of molecular targets according to various embodiments;

FIG. 2 shows a machine-learning modeling system for developing aptamers in accordance with various embodiments;

FIG. 3 shows a block diagram of a aptamer development platform according to various embodiments;

FIGS. 4A and 4B show an exemplary flow for aptamer development in accordance with various embodiments;

FIG. 5 shows an exemplary flow for aptamer development in accordance with various embodiments; and

FIG. 6 shows an exemplary computing device in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. Introduction

Identification of high affinity and high specificity binders (e.g., monoclonal antibodies, nucleic acid aptamers, and the like) of molecular targets (e.g., VEGF, HER2) has dramatically transformed treatment of many types of diseases (e.g., oncology, infectious disease, immune/inflammation, etc.). However, given the large search space of potential sequences (e.g., 10²⁴ potential sequences for the average aptamer or monoclonal antibody CDR-H3 binding loop) and the comparatively low-throughput of methodologies to assess the binding affinity of candidates (e.g., dozens to thousands per week) it is highly likely that optimal binder selection is not currently being achieved. While selection based approaches (e.g., phage display, SELEX, and the like) can potentially identify binders, among libraries of millions to trillions of candidates, there are several weaknesses with these approaches: (i) output is binary—it is challenging to know whether relatively strong binders in the library are actually strong binders; (ii) data is noisy—binding is dependent on every candidate encountering available target with the same relative frequency and variance from this can lead to many false negatives and some false positives; and (iii) capacity is much smaller than the total search space˜phage display (max candidates ˜10⁹) and SELEX (max candidates 10¹⁴) search spaces much smaller than the total possible search space (additionally, it is generally difficult (or expensive) to characterize the portions of the total sequence space that are searched).

To address these challenges, efforts have been made to apply computational and machine-learning techniques in an “experiment in the loop” process to reduce the search space and design better binders. For example, the following computational and machine-learning techniques have been attempted to increase discovery of viable high affinity/high specificity binders of molecular targets: (i) identification of libraries more likely to bind via prediction from physics based models, (ii) input of selection data and design/identify more likely binders (for monoclonal antibodies and nucleic acid aptamers), and (iii) address other factors beyond affinity that affect commercialization and therapeutic potential. To date however, these computational and machine-learning techniques have had limited success in designing markedly different sequences with better properties, let alone with sufficient predictive power to align on a small set of sequences appropriate for low-throughput characterization. Particularly, the techniques in the second category, often struggle to input sufficient data to identify or design candidates that are markedly different from the training sequences used to train the computation and machine-learning models.

To address these limitations and others, an aptamer development system is disclosed herein that derives in silico aptamers sequences from in vitro aptamer sequences found experimentally to bind to a target. For instance in an exemplary embodiment, an aptamer developmental process may comprise: obtaining initial sequence data for aptamers of an initial aptamer library that bind to a target, do not bind to the target, or a combination thereof; identifying, by a first machine-learning model, a first set of aptamer sequences as satisfying one or more constraints, where the first machine-learning model comprises model parameters learned from the initial sequence data, and the first set of aptamer sequences are derived from a subset of sequences from the initial sequence data, sequences from a pool of sequences different from sequences from the initial sequence data, or a combination thereof; obtaining, using an in vitro binding selection process, subsequent sequence data for aptamer of a subsequent aptamer library that bind to the target, do not bind to the target, or a combination thereof, where the subsequent aptamer library comprises aptamers synthesized from the first set of aptamer sequences; identifying, by a second machine-learning model, a second set of aptamer sequences as satisfying the one or more constraints, where the second machine-learning model comprises model parameters learned from the subsequent sequence data, and the second set of aptamer sequences are derived from a subset of sequences from the subsequent sequence data, sequences from a pool of sequences different from sequences from the subsequent sequence data, or a combination thereof; determining, using one or more in vitro assays, analytical data for aptamers synthesized from the second set of aptamer sequences; identifying a final set of aptamer sequences from the second set of aptamer sequences that satisfy the one or more constraints based on the analytical data associated with each aptamer; and outputting the final set of aptamer sequences.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent.

As used herein, the terms “a query” and “a given problem” are defined as a request from a user or an inquiry corresponding to an interaction between a target (e.g., a protein) and a molecule such as an aptamer. The terms are used interchangeably. The query or the given problem can include finding one or more aptamers that can bind or inhibit a target. The query or the given problem can include a particular number of aptamers to be found that can bind or inhibit a target. The query or the given problem can include particular interaction metrics between the aptamer and the target according to the nature of the query or the problem. For example, the query can include a particular binding affinity or inhibition rate as a results of the aptamer binding to the target. Accordingly, a solution to the given problem and a response to an inquiry are also used interchangeably. The response can include information corresponding to the target, the aptamer(s) found to bind or not bind the target (e.g., sequence data), the interaction metrics, or the number of aptamers in the response or solution.

As used herein, when an action is “based on” something, this means the action is based at least in part on at least a part of the something.

It will be appreciated that techniques disclosed herein can be applied to assess other biological material (e.g., other binders such as monoclonal antibodies) rather than aptamers. For example, alternatively or additionally, the techniques described herein may be used to assess the interaction between any type of biologic material (e.g., a whole or part of an organism such as E. coli, or a biologic product that is produced from living organisms, contain components of living organisms, or derived from human, animal, or microorganisms by using biotechnology) and a target, and derive another type of biologic material therefrom based on the assessment.

### II. End-to-End Pipeline to Identify and Generate Response to a Query

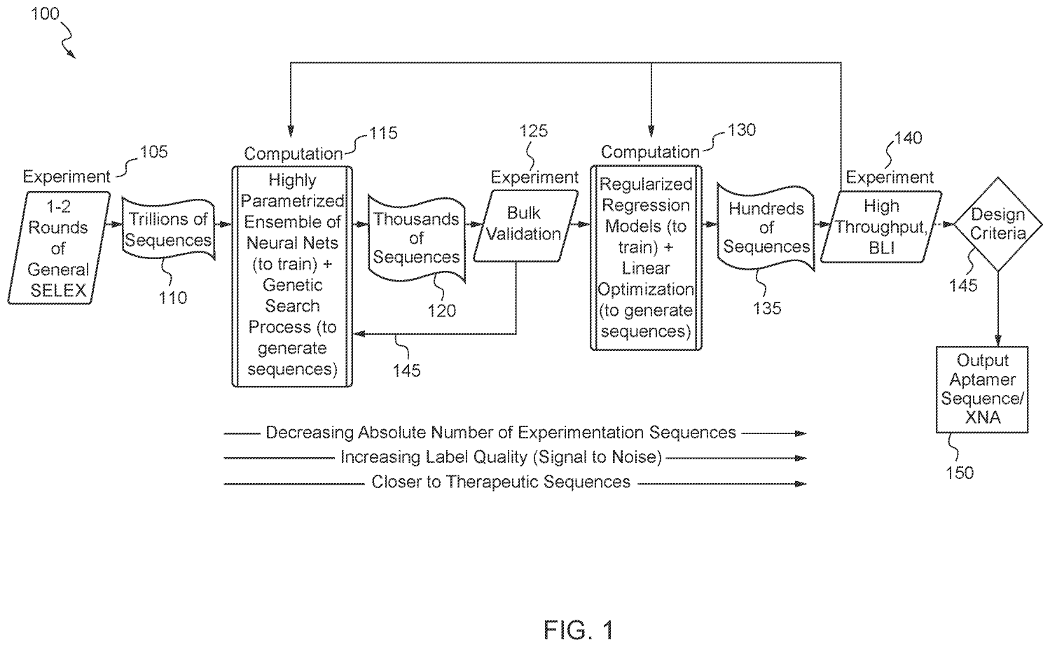

FIG. 1 shows a block diagram of a pipeline 100 for strategically identifying and generating high affinity binders of molecular targets. As used herein, the term “binding affinity” means the free energy differences between native binding and unbound states, which measures the stability of native binding states (e.g., a measure of the strength of attraction between an aptamer and a target). As used herein, a “high binding affinity” is a result from stronger intermolecular forces between an aptamer and a target leading to a longer residence time at the binding site (higher “on” rate, lower “off” rate). The factors that lead to high affinity binding include a good fit between surface of the molecules in their ground state and charge complementary (i.e., stronger intermolecular forces between the aptamer and the target). These same factors generally also provide a high binding specificity for the targets, which can be used to simplify screening approaches aimed at developing strong therapeutic candidates that can bind the given molecular target. As used herein, the term “binding specificity” means the affinity of binding to one target relative to the other targets. As used herein, the term “high binding specificity” means the affinity of binding to one target is stronger relative to the other targets. Various aspects described herein design and validate aptamers as strong therapeutic candidates that can bind the given molecular target based on binding affinity. However, it should be understood that design and validation of aptamers could involve the assessment of binding affinity and/or binding specificity.

Binding affinity can be measured or reported by the equilibrium dissociation constant (K_{D}), which is used to evaluate and rank order strengths of bimolecular interactions. The smaller the K_{D} value, the greater the binding affinity of the aptamer for its target. The larger the K_{D}value, the more weakly the target molecule and the aptamer are attracted to and bind to one another. In other words binding affinity and dissociation factor can have an inverse correlation. The strength of binding between an aptamer and its target can be also expressed by measuring or reporting a binding avidity between the aptamer and the target. While the term affinity characterizes an interaction between one aptamer domain with its binding site (assessed by corresponding dissociation constant K_{D}), the avidity refers to the overall strength of multiple binding interactions and can be described by the K_{D} of an aptamer-target complex.

In various embodiments, the pipeline 100 implements in vitro experiments and in silico computation and machine-learning based techniques to iteratively improve a process for identifying binders that can bind any given molecular target. At block 105, in vitro binding selections (e.g., phages display or SELEX) are performed where a given molecular target (e.g., a protein of interest) is exposed to tens of trillions of different potential binders (e.g., a library of 10¹⁴-10¹⁵ nucleic acid aptamers), a separation protocol is used to remove non-binding aptamers (e.g., flow-through), and the binding aptamers are eluted from the given target. The binding aptamers and/or the non-binding aptamers are sequenced to identify what aptamers do and/or do not bind the given target. This binding selection process may be repeated for any number of cycles (e.g., 1 to 3 cycles) to reduce the absolute count of potential aptamers from tens of trillions of different potential aptamers down to millions or trillions of sequences 110 of aptamers identified to have some level of binding (specific and non-specific) for the given target.

At block 115, the sequences of binding aptamers, non-binding aptamers, or a combination thereof obtained from block 105 are used to train a machine-learning algorithm (e.g., a highly parameterized machine algorithm with a parameter count of greater than or equal to 10,000, 30,000, 50,000, or 75,000) and learn a fitness function capable of ranking the fitness (quality) of sequences of aptamers based on one or more constraints such as a design criteria proposed for an aptamer, a problem being solved (e.g., finding an aptamer that is capable of binding to target with a high-affinity), and/or an answer to a query (e.g., what aptamers are capable of inhibiting function A). In some instances, the sequences of binding aptamers, non-binding aptamers, or a combination thereof are labeled with one or more sequence properties. The one or more sequence properties may include a binding-approximation metric that indicates whether a aptamer included in or associated with the training data bound to a particular target. The binding-approximation metric can include (for example) a binary value or a categorical value. The binding-approximation metric can indicate whether the aptamer bound to the particular target in an environment where the aptamer and other aptamers (e.g., other potential aptamers) are concurrently introduced to the particular target. The binding-approximation metric can be determined using a high-throughput assay, such as in vitro binding selections (e.g., phages display or SELEX), a low-throughput assay, such as in vitro Bio-Layer Interferometry (BLI), or a combination thereof. Additionally or alternatively, the one or more sequence properties may include a functional-approximation metric that indicates whether an aptamer included in or associated with the training data functions as intended (e.g., inhibits function A). The functional-approximation metric can include (for example) a binary value or a categorical value. The function-approximation metric can be determined using a low-throughput assay, such as an optical fluorescence assay or any other assay capable of detecting functional changes in a biological system (e.g., inhibiting an enzyme, inhibiting protein production, promoting binding between molecules, promoting transcription, etc.). Further, the function-approximation metric may be used to infer the binding-approximation metric (e.g., if function A is inhibited it can be inferred that the molecule bound to the particular target).

Machine-learning algorithms are procedures that are implemented in computer code and uses data (e.g., experimental results) to generate machine-learning models. The machine-learning models represent what was learned by the machine-learning algorithms during training. In other words, the machine-learning models are the data structures that are saved after running machine-learning algorithms on training data and represents the rules, variables, and any other algorithm-specific data structures required to make predictions. The use of a large data set with diverse sequences of binding aptamers (e.g., millions or trillions of binders) in the training allows for the algorithm to learn all of the parameters required for estimating the fitness of aptamer candidates for a given problem. Otherwise, the problem of having a large number parameters and dimensions yet small data sets results in overfitting, which means the learned function is too closely fit to a limited set of data points and works only for the data set the algorithm was trained with, rendering the learned parameters pointless.

The model in block 115 trained on the large data set from block 105 can then take new input sequences not necessarily discovered in an in vitro binding selection experiment and estimate a fitness for those input sequences given one or more constraints (e.g., finding an aptamer that is capable of binding to a given target with a high-affinity). The new input sequences may be generated using a machine-learning algorithm (e.g., an algorithm in block 115). In some instances, the machine-learning model in block 115 may include a genetic algorithm to generate new aptamers based on evolutionary models from one or more aptamers from binding selection experiments (e.g., from block 105). Thus, model(s) in block 115 may artificially increase the search space for aptamers that can bind the target and solve the given problem. The search space may be increased from the 10¹⁴-10¹⁵ nucleic acid aptamers investigated in the in vitro experimentation stage to at least 10²⁴ nucleic acid aptamers and beyond, depending on algorithm complexity and available computational resources.

The sequences of binding aptamers, non-binding aptamers, or a combination thereof obtained from block 105 are going to have a low signal to noise ratio (and low label quality). In other words, the sequences in 110 may include a small amount of sequences of aptamers with specific binding or high affinity (signal) and a large amount of aptamer sequences with non-specific binding or low affinity binding to the given target (noise). Essentially, the signal to noise ratio is a fraction of tested aptamers that have the desired binding characteristics when assayed with high/low throughput characterization or validation. Typically, machine-learning algorithms may model both signal and noise, or a relationship thereof. In other words, the model may include the two parts of the training data—the underlying generalizable truth (the signal), and the randomness specific to that dataset (the noise). Fitting both of those parts can increase the training set accuracy, but fitting the signal also increases test set accuracy or generalization (and real-world performance) while fitting the noise decreases both the test set accuracy and real-world performance (causes overfitting). Thus, conventional regularization techniques such as L1 (lasso regression), L2 (ridge regression), dropout, and the like may be implemented in the training to make it harder for the algorithm to fit the noise, and so more likely for the algorithm to fit the signal and generalize more accurately.

However, conventional regularization techniques can lead to dimensionality reduction, which means the machine-learning model is built using a lower dimensional dataset (e.g., less parameters). This can lead to a high bias error in the outputs (known as underfitting). In order to overcome these challenges and others, aspects of the present disclosure are directed to using a combination of in silico computational and machine-learning based techniques (e.g., ensemble of neural nets, genetic search processes, regularized regression models, linear optimization, and the like) in combination with various in vitro experimentation techniques (e.g., binding selections, SELEX, and the like) to identify or design markedly different sequences with better properties, while maintaining sufficient predictive power to align on a small set of sequences (e.g., tens to hundreds) appropriate for low-throughput characterization or validation.

These various machine-learning and experimentation techniques are implemented in the pipeline 100 via a aptamer development architecture (e.g., the exemplary architecture shown in FIG. 1 and described herein) to decrease the absolute number of sequences being used as input for each stage while increasing the signal to noise ratio (e.g., decreasing the noise) and label quality (e.g., quality of the binding-approximation metric and/or functional-approximation metric, and to ultimately predict the highest quality binders (e.g., highest-affinity) for any given molecular target. In some instances, a machine-learning algorithm used in block 115 is a series of algorithms such as a neural network (i.e., a parameter count of greater than or equal to 10,000, 30,000, 50,000, or 75,000). A series of algorithms offers increased flexibility and can scale in proportion to the amount of training data available. A tradeoff for this flexibility may be that the algorithms learn via a stochastic training algorithm which means that the algorithms are sensitive to both the specific training data set (presumed to be a random sample from some fixed distribution) and also the initial conditions, etc., of the training run (e.g., seeds for pseudo-random number generators). Additionally, there is also randomness that is hard to control for even if random seeds are set because modern GPUs (presumably TPUs) are not guaranteed deterministic. This means that the algorithms are subject to overfitting and can have high variance when it comes to making a final prediction (e.g., prediction of a fitness score for additional or alternative sequences of aptamers).

In order to overcome this variance, in some instances, the machine-learning algorithm is configured as a series of multiple neural networks trained using an ensemble based approach to combine the predictions from the multiple neural networks. Combining the predictions from multiple neural networks counters the variance of a single trained neural network model and can reduce generalization error (also known as the out-of-sample error, which is a measure of how accurately an algorithm is able to predict outcome values for previously unseen data). For example, generalization error is typically decomposed into bias and variance; bias is (roughly) reduced by more expressive models (e.g., neural nets with many more parameters) but increasing the flexibility of models can lead to overfitting. Variance is (roughly) reduced by ensembles or larger datasets. Thus, for instance, random forests are ensembles of very flexible models (decisions trees)—the low bias of the component models usually lead to high variance solutions, so this can be counteracted by using an ensemble of trees, each fit to a random subset (optionally along with other techniques) of the data. The results of the ensemble of neural networks are predictions that are less sensitive to the specifics of the training data, choice of training scheme, and the randomness inherent in a single training run.

The trained machine-learning model (e.g., an ensemble of neural networks) may then be used to perform a search process (e.g., genetic search) to identify sequences that have a high predicted fitness scores. The sequences of aptamers identified by the model(s) in block may then be output, as shown in block 120. The output 120 may comprise thousands of aptamer sequences. These sequences may be new sequences that could not be discovered using a binding selection experiment.

In some instances, the search process in the block 115 is a genetic search process that uses a genetic algorithm, which mimics the process of natural selection, where the fittest individuals (e.g., aptamers with a potential for binding a given target) are selected for reproduction in order to produce offspring of the next generation (e.g., aptamers with the greatest potential for binding the given target). If parents have better fitness, their offspring will be better than parents and have a better chance at surviving. This process may continue iterating until a generation with the fittest individuals are found. Therefore, the aptamers in block 120 (e.g., thousands of sequences) may have high probability or potential for satisfying the one or more constraints (e.g., binding the given target). In certain instances, the genetic algorithm is constrained to a limited number of nucleotide edits away from the training dataset knowing that variance of empirical labels relative to highly parameterized machine-learning model predictions increases drastically. The model may stop the search process when another criterion such as maximum number of predicted aptamers is met.

At block 125, identified or designed (e.g., by genetic search algorithm) sequences of aptamers 120 may be used to synthesize aptamers, which are used for subsequent binding selections. For example, subsequent in vitro binding selections (e.g., phages display or SELEX) may be performed where the given molecular target is exposed to the synthesized aptamers. A separation protocol may be used to remove non-binding aptamers (e.g., flow-through). The binding aptamers may then be eluted from the given target. The binding and/or non-binding aptamers may be sequenced to identify the sequence of aptamers that do and/or those that do not bind the given target. This binding selection process may be repeated for any number of cycles (e.g., 1 to 3 cycles) to validate which of the identified/designed aptamers from block 115 actually bind the given target. In some instances, the subsequent binding selections are performed using aptamers carrying Unique Molecular Identifiers (UMI) to enable accurate counting of copies of a given candidate sequence in elution or flow-through. Because the sequence diversity is reduced at this stage, there can be more copies of each aptamer to interact with the given target and improve the signal to noise ratio (and label quality).

The processes in blocks 105-125 may be performed once or repeated in part or in their entirety any number of times to decrease the absolute number of sequences and increase the signal to noise ratio, which ultimately results in a set of aptamer candidates that satisfy the one or more constraints (e.g., bind targets of interest in a inhibitory/activating fashion or to deliver a drug/therapeutic to a target such as a T-Cell). As used herein, “satisfy” the one or more constraints can be complete satisfaction (e.g., bound to the target), substantial satisfaction (e.g., bound to the target with an affinity above/below a given threshold or greater than 98% percent inhibition of a function A), or partial satisfaction (e.g., bound to the target at least 60% of the time or greater than 60% percent inhibition of a function A). The satisfaction of the constrain may be measured using one or more binding and/or analytical assays as described in detail herein. The output from block 125 (e.g., bulk validation) may include aptamers that can bind to the target with varying strengths (e.g., high, medium or low affinities). The output from block 125 may also include aptamers that are not be capable of binding to the target. In some instances, the sequences of binding aptamers, non-binding aptamers, or a combination thereof obtained from block 125 are used to improve the machine-learning models in block 115 (e.g., by retraining the machine-learning algorithms). The sequences of binding aptamers, non-binding aptamers, or a combination thereof from block 125 may be labeled with one or more sequence properties. As described herein, the one or more sequence properties may include a binding-approximation metric that indicates whether a aptamer included in or associated with the training data bound to a particular target and/or a functional-approximation metric that indicates whether a aptamer included in or associated with the training data functions as intended (e.g., inhibits function A). In certain instances, the binding-approximation metric is determined from the subsequent in vitro binding selections (e.g., phages display or SELEX) performed in block 125 and/or a low-throughput assay, such as in vitro BLI.

At block 130, the sequences of binding aptamers, non-binding aptamers, or a combination thereof (optionally labeled with one or more sequence properties) obtained from block 125 are used to train an algorithm to identify sequences of aptamers 135 that can satisfy the one or more constraints (e.g., bind a given target). The algorithm may identify hundreds of additional or alternative sequences. The algorithm may include linear algorithms (for example) a support vector algorithm/machine or a regression algorithm (e.g., a linear regression algorithm).

In some instances, the algorithm is a multiple regression algorithm. The regression algorithm may be trained using regularization techniques (i.e., fitting a model with more than one independent variable (covariates or predictors or features—all the same thing) to obtain a multiple regularized regression model. While the linear algorithms are less expressive than highly parametrized algorithms, the improved signal to noise at this stage can allow the linear algorithms to still capture signal while being better at generalizing.

Optimization techniques such as linear optimization may be used at this stage to identify the hundreds of additional or alternative sequences of aptamers 135 with differing relative fitness scores (and therefore affinity). Linear optimization (also called linear programming) is a computational method to achieve the best outcome (such as highest binding affinity for a given target) in a model whose requirements are represented by linear relationships (e.g., a regression model). More specifically, the linear optimization improves the linear objective function, subject to linear equality and linear inequality constraints to output the hundreds of additional or alternative sequences of aptamers 135 with differing relative fitness scores (including those with a highest binding affinity). Unlike the machine-learning model and searching process used in block 115, there is greater confidence in deviating away from training data in the process of linear optimization due to better generalization by the regression models. Consequently, the linear optimization may not be constrained to a limited number of nucleotide edits away from the training dataset.

At block 140, identified or designed aptamer sequences 135 may be used to synthesize new aptamers. These new aptamers may then be characterized or validated using experiments 140. The experiments may include high throughput binding selections (e.g., SELEX) or low-throughput assays. In some instances, the low-throughput assay (e.g., BLI) is used to validate or measure a binding strength (e.g., affinity, avidity, or dissociation constant) of an aptamer to the given target. In this context, BLI may include preparing a biosensor tip to include the aptamers in an immobilized form and a solution with the given target in a tip of a biosensor. Binding between the molecule(s) and the particular target increases a thickness of the tip of the biosensor. The biosensor is illuminated using white light, and an interference pattern is detected. The interference pattern and temporal changes to the interference pattern (relative to a time at which the molecules and particular target are introduced to each other) are analyzed to predict binding-related characteristics, such as binding affinity, binding specificity, a rate of association, and a rate of dissociation. In other instances, the low-throughput assay (e.g., a spectrophotometer to measure protein concentration) is used to validate or measure functional aspects of the aptamer such as its ability to inhibit a biological function (e.g., protein production).

The processes in blocks 105-140 may be performed once or repeated in part or in their entirety any number of times to decrease the absolute number of sequences and increase the signal to noise ratio, which ultimately results in a set of aptamer candidates that best satisfy the one or more constraints (e.g., bind targets of interest in a inhibitory/activating fashion or to deliver a drug/therapeutic to a target such as a T-Cell). The output from block 140 (e.g., BLI) may include aptamers that can bind to the target with varying strengths (e.g., high, medium or low affinities). The output from block 140 may also include aptamers that are not be capable of binding to the target. In some instances, the sequences of binding aptamers, non-binding aptamers, or a combination thereof obtained from block 140 are used to improve the machine-learning models in block 115 and/or 130 (e.g., by retraining the machine-learning algorithms). The sequences of binding aptamers, non-binding aptamers, or a combination thereof from block 140 may be labeled with one or more sequence properties. As described herein, the one or more sequence properties may include a binding-approximation metric that indicates whether a aptamer included in or associated with the training data bound to a particular target and/or a functional-approximation metric that indicates whether a aptamer included in or associated with the training data functions as intended (e.g., inhibits function A). In certain instances, the binding-approximation metric is determined from the subsequent in vitro BLI performed in block 140.

In block 145, a determination is made as to whether one or more of the aptamers evaluated in block 140 satisfy the one or more constraints such as the design criteria proposed for an aptamer, the problem being solved (e.g., finding an aptamer that is capable of binding to target with a high-affinity), and/or the answer to a query (e.g., what aptamers are capable of inhibiting function A). The determination may be made based on the binding-approximation metric and/or the functional-approximation metric associated with an aptamer satisfying the one or more constraints. In some instances, an aptamer design criteria may be used to select one or more aptamers to be output as the final solution to the given problem. For example, the design criteria in block 145 may include a binding strength (e.g., a cutoff value), a minimum affinity or avidity between the aptamer and the target, or a maximum dissociation constant.

In block 150, one or more aptamers from experiments 140 that are determined to satisfy the one or more constraints (e.g., showing affinity greater or equal to the minimum cutoff) are provided, for example, as the final solution to the given problem or as a result to a given query. The providing the output may include generating an output library comprising the final set of aptamers. The output library may be generated incrementally as new aptamers are generated and selected by performing and/or repeating blocks 105-145. At each repetition cycle one or more aptamers may be identified (i.e., designed, generated and/or selected) and added to the output based on their ability to satisfy the one or more constraints. The providing the output may further include transmitting the one or more aptamers or output library to a user (e.g., transmitting electronically via wired or wireless communication).

It will be appreciated that although FIG. 1 and the description herein, describe going from trillions of sequences to thousands of sequences to hundreds of sequences, these numbers are merely provided for illustrative purposes. In general, it should be understood that pipeline 100 is provisioned to start with a large data set (a large absolute number of experimentation sequences which could be, for example, septillions, trillions, billions, or millions) for training a highly-parametrized algorithm and eventually narrows down the absolute number of experimentation sequences to a more manageable number eventually aligning on a small data set (a small absolute number of experimentation sequences which could be, for example, hundreds, tens, or less) for low-throughput characterization and validation as potential therapeutic candidates.

### III. Modeling Systems to Identify/Design Aptamers that Bind or Inhibit a Target

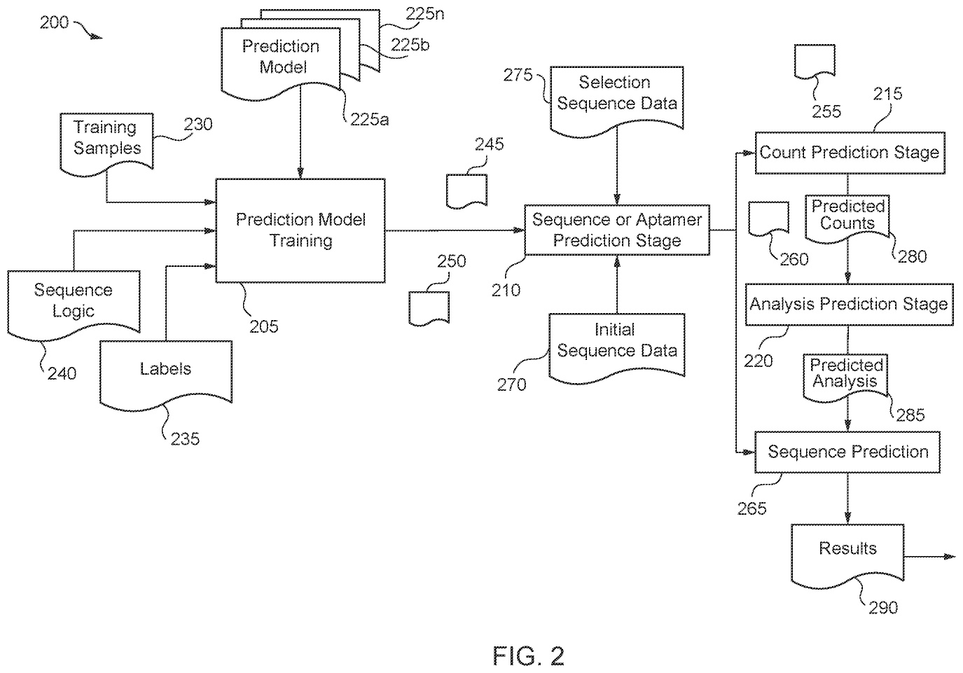

FIG. 2 shows a block diagram illustrating aspects of a machine-learning modeling system 200, such as the machine-learning algorithm 115. The a machine-learning modeling system 200 may be used for identifying or designing high affinity binders (e.g., aptamers, peptides, proteins, or peptidomimetics) of molecular targets. As shown in FIG. 2, the predictions performed by the machine-learning modeling system 200 in this example include several stages:

a prediction model training stage 205, one or more sequence or aptamer identification stages 210, an optional count prediction stage 215, and an optional analysis prediction stage 220.

The prediction model training stage 205 builds and trains one or more models 225a-225n (‘n’ represents any natural number) to be used by the other stages (which may be referred to herein individually as a model 225 or collectively as the models 225). For example, the models 225 can include one or more different type of models for generating sequences of aptamers not experimentally determined by a selection process but identified or designed (e.g., by a computational model) based on aptamers experimentally determined by a selection process. The models 225 may be used in the pipeline 100 described with respect FIG. 1 for identifying or designing high affinity binders for a given target. The models 225 can also include a model for predicting binding counts for the predicted sequences for derived aptamers. The models 225 can also include a model for predicting analytics such as binding affinity for the predicted sequences for derived aptamers. Still other types of prediction models may be implemented in other examples according to this disclosure.

A model 225 can be a machine-learning model, such as a neural network, a convolutional neural network (“CNN”), e.g. an inception neural network, a residual neural network (“ResNet”) or NASNet provided by GOOGLE LLC from MOUNTAIN VIEW, Calif., or a recurrent neural network, e.g., long short-term memory (“LSTM”) models or gated recurrent units (“GRUs”) models. A model 225 can also be any other suitable machine-learning model trained to predict predicted sequences for derived aptamers, sequence counts or analytics for aptamer sequences, such as a support vector machine, decision tree, a three-dimensional CNN (“3DCNN”), regression model, linear regression model, ridge regression model, logistic regression model, a dynamic time warping (“DTW”) technique, a hidden Markov model (“HMM”), etc., or combinations of one or more of such techniques—e.g., CNN-HMI or MCNN (Multi-Scale Convolutional Neural Network). The machine-learning modeling system 200 may employ one or more of same type of model or different types of models for aptamer sequence prediction, aptamer count prediction, and/or analysis prediction.

To train the various models 225 in this example, training samples 230 for each model 225 are obtained or generated. The training samples 230 for a specific model 225 can include the sequence data as described with respect to FIG. 1 and optional labels 235 corresponding to the sequence data. For example, for a model 225 to be utilized to identify or design an aptamer sequence, the input can be the aptamer sequence itself or features extracted from the sequence data associated with the aptamer sequence and optional labels 235 can include a binding-approximation metric, functional-approximation metric, and/or calculated fitness scores (a measure of how well each aptamer sequences satisfies one or more constraints) for the aptamer sequences. Similarly, for a model 225 to be utilized to predict a count or binding affinity for an aptamer sequence, the input can include the sequence and count features extracted from the initial sequence data and/or the sequence data associated with the sequence, and the optional labels 235 can include features indicating parameters for the count, binding affinity, or functional analysis (e.g., a binding approximation metric and/or functional-approximation metric) or a vector indicating probabilities for the count, binding affinity, functional analysis of the sequence data.

In some instances, the training process includes iterative operations to find a set of parameters for the model 225 that maximizes or minimizes an objective function (e.g., regression or classification loss) for the models 225. Each iteration can involve finding a set of parameters for the model 225 so that the value of the objective function using the set of parameters is smaller or greater than the value of the objective function using another set of parameters in a previous iteration. The objective function can be constructed to measure the difference between the outputs predicted using the models 225 and the optional labels 235 contained in the training samples 230. Once the set of parameters are identified, the model 225 has been trained and can be tested, validated, and/or utilized for prediction as designed.

In addition to the training samples 230, other auxiliary information can also be employed to refine the training process of the models 225. For example, sequence logic 240 can be incorporated into the prediction model training stage 205 to ensure that the sequences or aptamers, counts, and analysis predicted by a model 225 do not violate the sequence logic 240. For example, binding affinity (the strength of the binding interaction between an aptamer and a target) is a characteristic that can drive aptamers to be present in greater numbers in a pool of aptamer-target complexes after a cycle of selection process. This relationship can be expressed in the sequence logic 240 such that as the binding affinity variable increases the predictive count increases (to represent this characteristic), as the binding affinity variable decreases the predictive count decreases. Moreover, an aptamer sequence generally has inherent logic among the different nucleotides. For example, GC content for an aptamer is typically not greater than 60%. This inherent logical relationship between GC content and aptamer sequences can be exploited to facilitate the aptamer sequence prediction.

According to some aspects of the disclosure presented herein, the logical relationship between the binding affinity and count can be formulated as one or more constraints to the optimization problem for training the models 225. A training loss function that penalizes the violation of the constraints can be built so that the training can take into account the binding affinity and count constraints. Alternatively, or additionally, structures, such as a directed graph, that describe the current features and the temporal dependencies of the prediction output can be used to adjust or refine the features and predictions of the models 225. In an example implementation, features may be extracted from the initial sequence data and combined with features from the selection sequence data as indicated in the directed graph. Features generated in this way can inherently incorporate the temporal, and thus the logical, relationship between the initial library and subsequent pools of aptamer sequences after cycles of the selection process. Accordingly, the models 225 trained using these features can capture the logical relationships between sequence characteristics, selection cycles, aptamer sequences, and nucleotides.

Although the training mechanisms described herein mainly focus on training a model 225, these training mechanisms can also be utilized to fine tune existing models 225 trained from other datasets. For example, in some cases, a model 225 might have been pre-trained using pre-existing aptamer sequence libraries. In those cases, the models 225 can be retrained using the training samples 230 containing initial sequence data, experimentally derived selection sequence data, and other auxiliary information as discussed herein.

The prediction model training stage 205 outputs trained models 225 including trained nonlinear or highly parametrized models 245, trained linear models or models with minimal parameters 250, optionally trained count prediction models 255, and optionally trained analysis prediction models 260. The trained nonlinear or highly parametrized models 245 and trained linear models or models with minimal parameters 250 may be used in the sequence identification stages 210 to identify or design sequences 265 based on a subset or all of the initial sequence data 270 (e.g., random sequence data), the selection sequence data 275 identified during the experimental selection process (e.g., blocks 105-140 described with respect to FIG. 1), or a combination thereof. The trained count prediction models 255 may be used in the count prediction stage 215 to generate count predictions 280 for the identified sequences based on the initial sequence data 270 and/or the selection sequence data 275 identified during the experimental selection process (e.g., blocks 105, 125, and 140 described with respect to FIG. 1). The trained analysis prediction models 260 may be used in the analysis prediction stage 220 to generate analysis predictions 285 (e.g., a binary classifier such as binds to target or does not bind to target) for the identified sequences based on the initial sequence data 270 and/or the selection sequence data 275 identified during the experimental selection process (e.g., blocks 105, 125, and 140 described with respect to FIG. 1). In some instances, the identified or designed sequences 265, count predictions 280, analysis predictions 285, or any combination thereof may be provided as results 290.

The results 290 may be used to synthesize aptamers to be validate or improved experimentally. The results 290 may be a solution to a given problem or a query (e.g., posed by a user). For example, in response to a query for top hundred aptamers that bind a given target, the results 290 may include the identity of sequences for a hundred aptamers with the highest count or binding affinity for the given target. As described with respect to FIG. 1, the results 290 may then be used to synthesize the aptamers to be used in low-throughput assays for characterizing or validating the results 290 as potential therapeutic candidates.

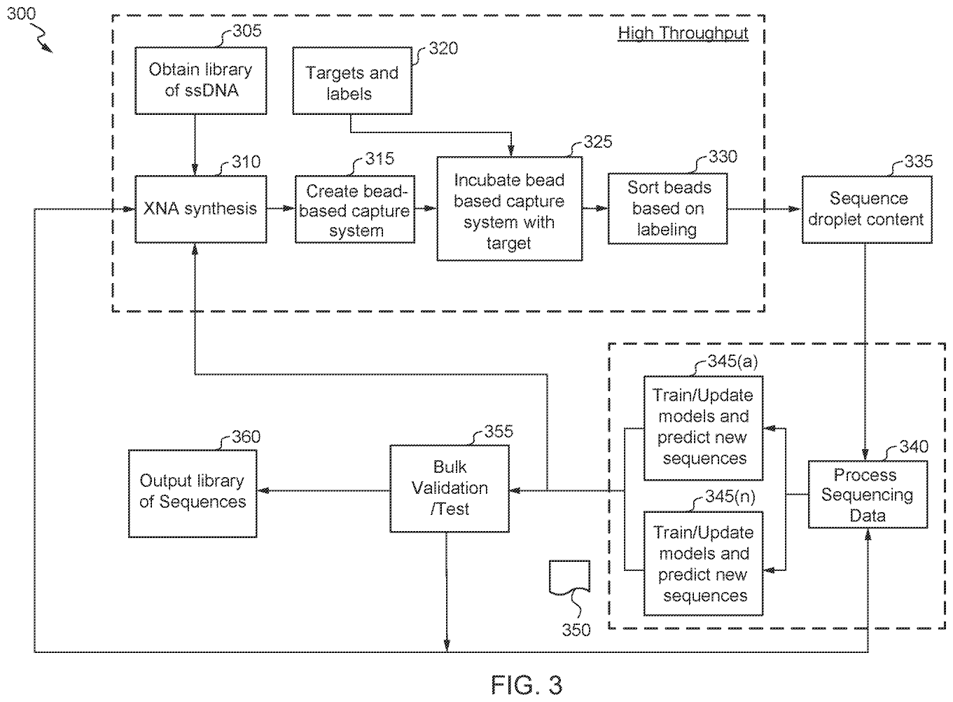

FIG. 3 shows a block diagram of an aptamer development platform 300 for strategically identifying and generating high affinity binders of molecular targets. In various embodiments, the aptamer development platform 300 implements in vitro experiments and in silico computation and machine-learning based techniques to iteratively improve a process for identifying binders that can bind any given molecular target. The various components of the aptamer development platform 300 are executed in accordance with the pipeline developed for identifying and generating high affinity binders of molecular targets (as described with respect to FIG. 1). The in silico computation and machine-learning based techniques are trained and deployed as at least part of a machine-learning modeling system (as described with respect to FIG. 2).

In various embodiments, the aptamer development platform 300 implements screening-based techniques for aptamer discovery where each candidate aptamer sequence in a library is assessed based on a query or a problem (e.g., binding affinity with one or more targets or functionally capable of inhibiting one or more targets) in a high throughput binding selection process. As described herein, the aptamer development platform 300 implements machine-learning based techniques for enhanced aptamer discovery where candidate aptamer sequences in a library that satisfy the query are used to train one or more machine-learning models to identify additional or alternative candidate aptamer sequences that potentially satisfy the query. In some cases, the candidate aptamer sequences provided to platform 300 may comprise aptamers that are not capable of binding to a target (non-binders) The platform 300 may generate aptamers that satisfy the query based on the sequence of non-binders. The additional or alternative candidate aptamers may not be discovered by conventional methods. The query may comprise one or more design criteria such as binding affinity, avidity, or a dissociation constant of an aptamer and its target.

The aptamer development platform 300 further implements screening-based techniques for aptamer validation to validate or confirm that the identified aptamer candidate sequences do satisfy the query (e.g., bind to or inhibit the one or more targets) in a high throughput or low throughput manner. As should be understood, these techniques from screening through identification to validation can be repeated in one or more closed loop processes sequentially or in parallel to ultimately assess any number of queries.

The aptamer development platform 300 includes obtaining one or more single stranded DNA (deoxyribonucleic acid) or RNA (ribonucleic acid) (ssDNA [single-stranded DNA] or ssRNA [single-stranded RNA]) libraries at block 305. The one or more ssDNA or ssRNA libraries may be obtained from a third party (e.g., an outside vendor) or may be synthesized in-house. Each of the one or more libraries typically contains up to 10¹⁷ different unique sequences.

At block 310, the ssDNA or ssRNA of the one or more libraries are transcribed to synthesize a Xeno nucleic acid (XNA) aptamer library. XNA aptamer sequences (e.g., threose nucleic acids [TNA], 1,5-anhydrohexitol nucleic acid [HNA], cyclohexene nucleic acid [CeNA], glycol nucleic acid [GNA], locked nucleic acid [LNA], peptide nucleic acid [PNA], FANA [fluoro arabino nucleic acid]) are synthetic nucleic acid analogues that have a different sugar backbone than the natural nucleic acids DNA and RNA. XNA may be selected for the aptamer sequences as these polymers are not readily recognized and degraded by nucleases, and thus are well-suited for in vivo applications. XNA aptamer sequences may be synthesized in vitro through enzymatic or chemical synthesis. For example, an XNA library of aptamers may be generated by primer extension of some or all of the oligonucleotide strands in a ssDNA library, flanking the aptamer sequences with fixed primer annealing sites for enzymatic amplification, and subsequent PCR amplification to create an XNA aptamer library that includes 10¹²-10¹⁷ aptamer sequences.

In some instances, the XNA aptamer library may be processed for application in downstream machine-learning processes. In certain instances, the aptamer sequences are processed for use as training data, test data, or validation data in one or more machine-learning models. In other instances, the aptamer sequences are processed for use as actual experimental data in one or more trained machine-learning models. In either instance, the aptamer sequences may be processed to generate initial sequence data comprising a representation of the sequence of each aptamer and optionally a count metric. The representation of the sequence can include one-hot encoding of each nucleotide in the sequence that maintains information about the order of the nucleotides in the aptamer. The representation of the sequence can additionally or alternatively include a string of category identifiers, with each category representing a particular nucleotide. The count metric can include a count of each aptamer in the XNA aptamer library.

At block 315, the aptamers within the XNA aptamer library are partitioned into monoclonal compartments (e.g., monoclonal beads or compartmentalized droplets) for high throughput aptamer selection. For example, the aptamers may be attached to beads to generate a bead-based capture system for a target. Each bead may be attached to a unique aptamer sequence generating a library of monoclonal beads. The library of monoclonal beads may be generated by sequence-specific partitioning and covalent attachment of the sequences to the beads, which may be polystyrene, magnetic, glass beads, or the like. In some instances, the sequence-specific partitioning includes hybridization of XNA aptamers with capture oligonucleotides having an amine modified nucleotide for interaction with covalent attachment chemistries coated on the surface of a bead. In certain instances, the covalent attachment chemistries include N-hydroxysuccinimide (NHS) modified PEG, cyanuric chloride, isothiocyanate, nitrophenyl chloroformate, hydrazine, or any combination thereof. In some instances, UMIs are attached to the aptamers to enable accurate counting of copies of a given candidate sequence in elution or flow-through.

At block 320, a target (e.g., proteins, protein complexes, peptides, carbohydrates, inorganic molecules, cells, etc.) is obtained. The target may be obtained as a result of a query posed by a user (e.g., a client or customer). For example, a user may pose a query concerning identification of a hundred aptamers with the highest binding affinity for a given target or twenty aptamers with the greatest ability to inhibit activity of a given target. In some instances, the target is tagged with a label such as a fluorescent probe. At block 325, the bead-based capture system is incubated with the labeled target to allow for the aptamers to bind with the target and form aptamer-target complexes.

At block 330, the beads having aptamer-target complexes are separated from the beads having non-binding aptamers using a separation protocol. In some instances, the separation protocol includes a fluorescence-activated cell sorting system (FACS) to separate the beads having the aptamer-target complexes from the beads having non-binding aptamers. For example, a suspension of the bead-based capture system may be entrained in the center of a narrow, rapidly flowing stream of liquid. The flow may be arranged so that there is separation between beads relative to their diameter. A vibrating mechanism causes the stream of beads to break into individual droplets (e.g., one bead per droplet). Before the stream breaks into droplets, the flow passes through a fluorescence measuring station where the fluorescent label which is part of the aptamer-target complexes is measured. An electrical charging ring may be placed at a point where the stream breaks into droplets. A charge may be placed on the ring based on the prior fluorescence measurement, and the opposite charge is trapped on the droplet as it breaks from the stream. The charged droplets may then fall through an electrostatic deflection system that diverts droplets into containers based upon their charge (e.g., droplets having beads with aptamer-target complexes go into one container and droplets having beads with non-binding aptamers go into a different container). In some instances, the charge is applied directly to the stream, and the droplet breaking off retains a charge of the same sign as the stream. The stream may then returned to neutral after the droplet breaks off.

At block 335, the aptamers from the aptamer-target complexes are eluted from the beads and target, and amplified by enzymatic or chemical processes to optionally prepare for subsequent rounds of selection (repeat blocks 310-330, for example a SELEX protocol). The stringency of the elution conditions can be increased to identify the tightest-binding or highest affinity sequences. In some instances, once the aptamers are separated and amplified, the aptamers may be sequenced to identify the sequence and optionally a count for each aptamer.

Optionally, the separated non-binding aptamers are amplified by enzymatic or chemical processes. In some instances, once the non-binding aptamers are amplified, the non-binding aptamers may be sequenced to identify the sequence and optionally a count for each non-binding aptamer. The sequence and count of non-binding aptamers may provide information on which aptamers have the weakest binding (e.g., may be used in training of a machine-learning model), which may supplement or validate the results of the aptamers found to bind. If aptamers are high in count for non-binding and low in count for binding, then aptamers may be determined and validated to have a weak binding affinity. If certain aptamers have significant counts for both binding and non-binding, the aptamers may be limited for some other reason (e.g., competition for binding sites among same type of aptamers).

At block 340, a data set including the sequence, the count, and/or an analysis performed based on the separation protocol (e.g., a binary classifier or a multiclass classifier) for each aptamer that has gone through the selection process of steps 310-330 is processed for application in downstream machine-learning processes. The processing is performed by a controller/computer of platform 300. The data set may include the sequence, the count, and/or the analysis from the binding aptamers (those that formed the aptamer-target complexes), the non-binding aptamers (those that did not form the aptamer-target complexes), or the combination thereof. In general, there are different types of binders (e.g., agonist, antagonist, allosteric, etc.) and those would be characteristics that the system may be configured to distinguish between the different types of binders during training, testing, and/or experimental analysis. In some instances, the sequence, count, and/or analysis for each aptamer is processed for use as training data, test data, or validation data in one or more machine-learning models. In other instances, the sequence, count, and/or analysis for each aptamer is processed for use as actual experimental data in one or more trained machine-learning models. In either instance, the sequence, count, and/or analysis for each aptamer may be processed to generate selection sequence data comprising a representation of the sequence of each aptamer, a count metric, an analysis metric, or any combination thereof.

The representation of the sequence can include one-hot encoding of each nucleotide in the sequence that maintains information about the order of the nucleotides in the aptamer. The representation of the sequence can additionally or alternatively include other features concerning the sequence and/or aptamer, for example, post-translational modifications, binding sites, enzyme active sites, local secondary structure, kmers or characteristics identified for specific kmers, etc. The representation of the sequence can additionally or alternatively include a string of category identifiers, with each category representing a particular nucleotide. The count metric may include a count of the aptamer detected subsequent to an exposure to the target (e.g., during incubation and potentially in the presence of other aptamers). In some instances, the count metric includes a count of the aptamer detected subsequent to an exposure to the target in each round of selection. The analysis metric may include the binding-approximation metric, functional-approximation metric, and/or calculated fitness scores. For example, the analysis metric may include a binary classifier such as functionally inhibited the target, functionally did not inhibit the target, bound to the target, or did not bound to the target, a fitness score, which is a measure of how well a given aptamer sequence performs as a solution with respect to the given problem, and/or a multiclass classifier such as a level of functional inhibition or a gradient scale for binding affinity.

At blocks 345a-n, one or more machine-learning algorithms are trained by the controller/computer using the initial sequence data (from block 310), the selection sequence data (from block 335), or a combination thereof processed in block 340 as a data set (e.g., a training data set) to generate one or more trained machine-learning models. The one or more machine-learning models may include supervised models such as regression models (e.g., linear, decision tree, random forest, neural networks, etc.) or classification models (e.g., logistic regression, support vector machine, decision tree, random forest, neural networks, etc.) or unsupervised models such as clustering models (e.g., k-means, density-based, mean shift, etc.) or dimensionality reduction models (e.g., principle component analysis, etc.). In some instances (e.g., 345(a)), the machine-learning models include a neural network such as a feedforward neural network, a recurrent neural network, a convolutional neural network, or an ensemble of neural networks. In other instances, (e.g., 345(b)), the machine-learning models include a linear model such as a regression model or a regularized regression model. The machine-learning algorithms may be trained using training data, test data, and validation data based on sets of initial sequence data and selection sequence data to predict fitness scores and identify aptamer sequences (e.g., aptamers not experimentally determined by a selection process but identified based on aptamers experimentally determined by a selection process) and optional counts and/or analytics for the identified aptamer sequences. An objective function or loss function, such as a Mean Square Error (MSE), likelihood loss, or log loss (cross entropy loss), may be used to train each of the one or more machine-learning models. In some instances, a machine-learning algorithm may be trained for predicting fitness scores and identifying aptamer sequences using the initial sequence data and/or the selection sequence data. Another machine-learning algorithm may be trained for predicting binding counts for the identified aptamer sequences using the initial sequence data and/or the selection sequence data. Another machine-learning algorithm may be trained for predicting analytics such as binding affinity for the identified aptamer sequences using the initial sequence data and/or the selection sequence data.

The trained machine-learning models are then used to predict fitness scores and identify aptamer sequences and optional counts and/or analytics for the identified aptamer sequences. For example, a subset of the aptamers experimentally determined by the selection process to satisfy the query (e.g., aptamers that have high binding affinity with a target or predicted counts due primarily to high binding affinity with a target) can be identified and separated from aptamers experimentally determined by the selection process to not satisfy the query (non-binders). The sequences for the subset of aptamers experimentally determined by the selection process to satisfy the query (binders), sequences from a pool of sequences (e.g., a random pool of sequences or sequences pooled from a related library of sequences) different from the sequences from the subset of aptamers experimentally determined by the selection process, or a combination thereof can then be input into one or more machine-learning models to predict fitness scores and identify in silico derived aptamer sequences (e.g., aptamer sequences that are derivatives of the experimentally selected aptamers) and optionally counts and analytics for the derived aptamer sequences. Optionally, the subset of the aptamers experimentally determined by the selection process that do not satisfy the query can also be input into one or more machine-learning models to assist in identifying in silico derived aptamer sequences (e.g., aptamer sequences that are derivatives of the experimentally selected aptamers) and optionally counts and analytics for the derived aptamer sequences.

In some instances, additional techniques including the application of one or more different types of algorithms such as search algorithms (e.g., a genetic algorithm) or optimization algorithms (e.g., linear optimization) are used in combination with the one or more machine-learning models to improve upon the identification or design of aptamer sequences. For example, a subset of the aptamers experimentally determined by the selection process to satisfy the query can be identified and separated from aptamers experimentally determined by the selection process to not satisfy the query. This subset of aptamers, sequences from a pool of sequences different from the sequences in the subset of aptamers experimentally determined by the selection process, or a combination thereof may be used in a genetic search process that implements the trained machine-learning models as a learned fitness function for a genetic algorithm. The subset of aptamers can be input into the trained machine-learning models, which are used to predict fitness scores and identify in silico aptamer sequences for mating. Additionally, the trained machine-learning models (e.g., an ensemble off neural networks) may be configured to provide an uncertainty score regarding the predicted fitness score of a aptamer sequence as a binder, and the uncertainty score can be used in the genetic search process as at least part of a fitness score or as a filter for each identified aptamer sequence. The uncertainty score is determined using an uncertainty quantification process (e.g., a Gaussian process, a Monte Carlo dropout, non-Bayesian type processes, and the like) that quantifies uncertainty for predictions of the trained machine-learning models.

In the genetic algorithm, the subset of sequences experimentally determined by the selection process to satisfy the query, sequences from a pool of sequences different from the sequences from the subset of aptamers experimentally determined by the selection process, or a combination thereof serve as the initial population and a fitness function (i.e., the trained machine-learning model(s)) may be used to determine how fit each aptamer sequence is (e.g., the ability of each sequence to compete as a binder with other sequences). The fitness function estimates or predicts a fitness score for each sequence. The probability that each sequence will be selected for reproduction is based on its fitness score and optionally may take into consideration the uncertainty score generated by trained machine-learning models for each predicted fitness score. Thereafter, pairs of sequences are selected based on their fitness scores. Sequences with high fitness have more chance to be selected for reproduction. Offspring are created by exchanging the genes (e.g., nucleotides) of parent sequences among themselves until a crossover point is reached. The new offspring are added to the population, and the process may be repeated until the population has converged (does not produce offspring which are significantly different from the previous generation). Then it may be determined that the genetic algorithm has identified or designed a set of solutions or sequences for binding to the given target. In certain instances, certain new offspring formed can be subjected to a mutation with a low random probability. This means that some of the nucleotides in the sequence can be randomly changed. In some instances, the genetic algorithm is constrained to control the cross over point and/or the mutations to a limited number of edits away from the training dataset.

At block 350, the output of the trained machine-learning models (identified aptamer sequences, fitness scores, and optional counts and/or analytics of the identified aptamer sequences) may trigger recording of some or all of the in silico identified aptamer sequences (e.g., positive and negative aptamer data such as predicted counts demonstrating increased binding affinity for a target or predicted counts demonstrating decreased binding affinity for a target) within a data structure (e.g., a database table). In some instances, the identified aptamer sequences are recorded in a data structure in association with additional information including the query (i.e., the given problem), the one or more targets that are the focus of the query and basis for the identification of the aptamer sequences, counts predicted for the aptamer sequences, fitness scores, analysis predicted for the aptamer sequences, or any combination thereof.

Additionally or alternatively, the output of the trained machine-learning models may trigger subsequent binding selections at blocks 310-335, or experimental testing or validation at block 355 to select aptamers that satisfy the one or more constraints (e.g., have a high selectivity or specificity to a target).

At block 355, experimental testing or validation is performed on some or all of the in silico aptamer sequences to experimentally measure analytics such as binding affinities with the target and/or functional aspects with respect to the target. The experimental testing may be conditioned on input from a user. For example, a user device may present an interface in which the in silico aptamer sequences are identified along with input components configured to receive input to modify the in silico aptamer sequences (e.g., by removing or adding aptamers) and/or to generate an experiment-instruction communication to be sent to another device and/or other system. The experiment can include synthesizing each of the in silico aptamer sequences. These aptamers can then be validated in the wet lab in either individual or bulk experiments using low throughput or high throughput assays. For example, the user can access a single aptamer (e.g. oligonucleotide). The single aptamer can be provided by an aptamer source, such as Twist Biosciences, Agilent, IDT, etc. The aptamer can be used to conduct biochemical assays (e.g. gel shift, surface plasma resonance, bio-layer interferometry, etc.). In some instances, multiple aptamers in a singular pool can be used to rerun the equivalent SELEX protocol (e.g., blocks 310-335) to identify enriched aptamers. Results can be assessed to determine whether the computational experiments are verified. In some instances, selections can be run in a digital format (i.e., ones that give a functional output per sequence) to validate particular sequences. In some cases, the results may be used to improve the models in 345(a)-(n). For example, the validated sequences and metrics thereof can be used to update the training set and retrain the models in 345(a)-(n).

At block 360, the validated sequences and metrics thereof from block 355 may be used to generate or curate a final set of aptamers that satisfy the one or more constraints (e.g., as a final solution to a given problem). For example, an output library comprising a final set of aptamers may be generated. The output library may be generated incrementally as new aptamers are generated and selected by repeating different steps in the aptamer development platform 300 (e.g., several cycles of 300 in a loop). At each repetition cycle one or more aptamers may be designed, generated and/or selected and added to the output 260 based on their ability to satisfy the one or more constraints.

Overall, in accordance with pipeline 100 illustrated in FIG. 1, the aptamer development platform 300 may perform: (i) a first round of binding selections at blocks 305-335, (ii) processing and input of derived aptamers into a first trained machine-learning model (e.g., an ensemble of neural networks) at blocks 340 and 345(a), (iii) a validation test at block 355 which may include a second round of binding selections at blocks 310-335 (followed optionally by retraining of the first trained machine-learning model), (iv) processing and input of derived aptamers into a second trained machine-learning model (e.g., a regression model) at blocks 340 and 345(b), and (v) experimental testing or validation at block 355 (followed optionally by retraining of the first and/or second trained machine-learning model) to facilitate the identification of aptamers at block 360 that satisfy the one or more constraints.

As should be understood, the aptamer development platform 300 described with respect to FIG. 3 could be used for aptamer discovery where steps 310-335 are run in parallel to generate multiple monoclonal beads against multiple targets in association with one or more queries. Additionally or alternatively, the aptamer development platform 300 described with respect to FIG. 3 could be used for aptamer discovery where steps 310-335 are run in parallel to generate multiple monoclonal beads against multiple targets in association with one or more queries and identify in parallel aptamer sequences and optional counts and/or analytics for the identified aptamer sequences.

The machine-learning models trained and used to make the predictions may be updated with results from the experiments and other machine-learning models using a distributed or collaborative learning approach such as federate learning which trains machine-learning models using decentralized data residing on end devices or systems. For example, a central or primary model may be updated (i.e., retrained or improved) or trained with results from all experiments being run and the results of the updating/training of the central or primary model may be propagated through to deployed secondary models (e.g., if information is obtained on cytokine a then the system may use that information to potential refine processes to identify for cytokine b).

### IV. Modeling Processes and Techniques to Identify or Design Aptamers

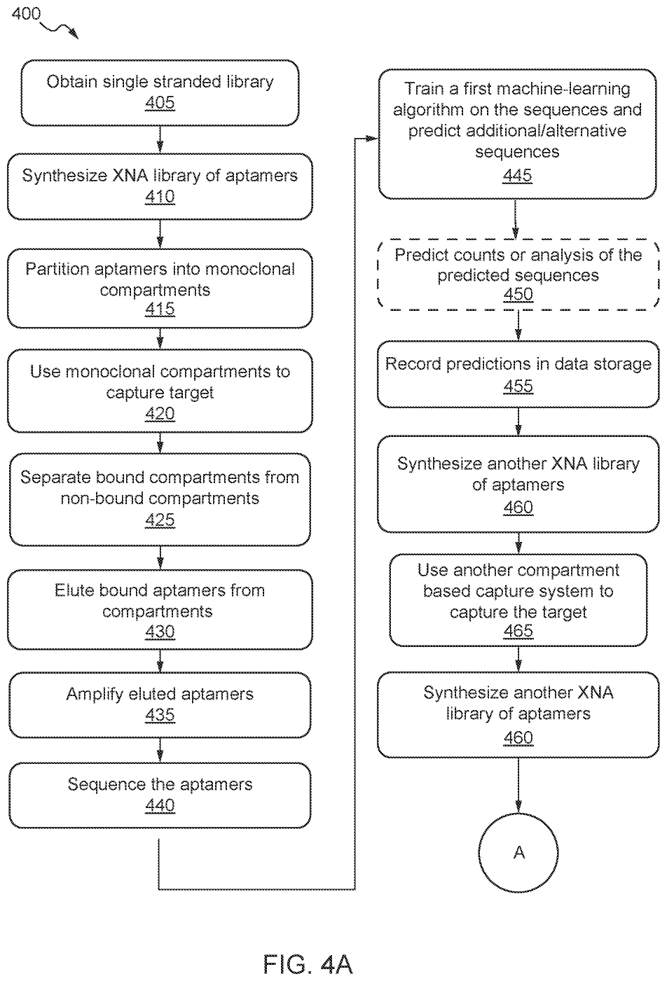

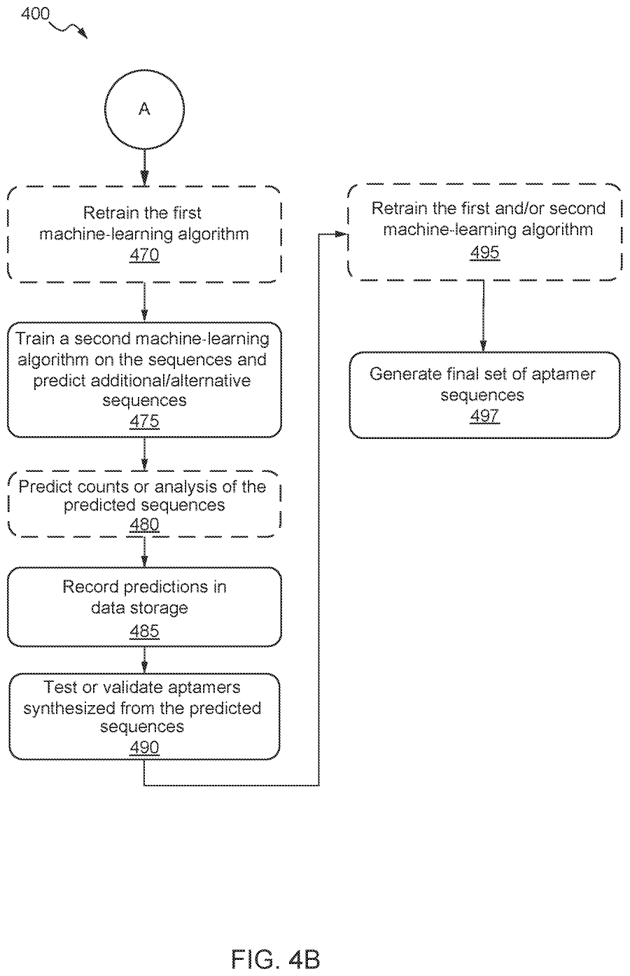

FIGS. 4A and 4B are a simplified flowchart 400 illustrating an example of processing for developing aptamers using a machine-learning modeling system and an aptamer development platform (e.g., machine-learning modeling system 200 and the aptamer development platform 300 described with respect to FIGS. 2 and 3). Process 400 begins at block 405, at which one or more single stranded DNA or RNA (ssDNA or ssRNA) libraries are obtained. The one or more ssDNA or ssRNA libraries comprise a plurality of ssDNA or ssRNA sequences. At block 410, an XNA aptamer library is synthesized from the one or more ssDNA or ssRNA libraries. The XNA aptamer sequences that make up the XNA aptamer library may be synthesized in vitro with a transcription assay that includes enzymatic or chemical synthesis. The XNA aptamer library comprises a plurality of aptamer sequences. It will be appreciated that techniques disclosed herein can be applied to assess other aptamers rather than XNA aptamers. For example, alternatively or additionally, the techniques described herein may be used to assess the interactions between any type of sequence of nucleic acids (e.g., DNA and RNA) and epitopes of a target. Thus, the following block may synthesize a DNA or RNA aptamer library as input for aptamer sequences rather than constructing an XNA library.

At block 415, the plurality of aptamers within the XNA aptamer library (optionally DNA or RNA libraries) are partitioned into monoclonal compartments that combined establish a compartment-based capture system. Each monoclonal compartment comprises a unique aptamer from the plurality of aptamers. In some instances, the one or more monoclonal compartments are one or more monoclonal beads. In some instances, each monoclonal compartment or unique aptamer comprises a unique barcode (e.g., a unique molecular identifiers such as a unique sequence of nucleotides) for tracking identification of the compartment and/or the aptamer associated with the monoclonal compartment. At block 420, the compartment-based capture system is used to capture one or more targets. The capturing comprises the one or more targets binding to the unique aptamer within one or more monoclonal compartments. In some instances, the one or more targets are identified based on a query received from a user. As used herein, when an action is “based on” something, this means the action is based at least in part on at least a part of the something. At block 425, the one or more monoclonal compartments of the compartment-based capture system that comprise the one or more targets bound to the unique aptamer are separated from a remainder of monoclonal compartments of the compartment-based capture system that do not comprise the one or more targets bound to a unique aptamer. In some instances, the one or more monoclonal compartments are separated from the remainder of monoclonal compartments using a fluorescence-activated cell sorting system.

At block 430, the unique aptamer is eluted from each of the one or more monoclonal compartments and/or the one or more targets. At block 435, the unique aptamer from each of the one or more monoclonal compartments is amplified by enzymatic or chemical processes. At block 440, the unique aptamer from each of the one or more monoclonal compartments (e.g., the bound aptamers) are sequenced. The sequencing comprises using a sequencer (and optionally an additional assay such as BLI or a spectrometer) to generate sequencing data and optionally analysis data (e.g., a binding-approximation metric and/or functional-approximation metric) for the unique aptamer from each of the one or more monoclonal compartments. The analysis data for the unique aptamer from each of the one or more monoclonal compartments may indicate the unique aptamer did bind to the one or more targets. In some instances, the sequencing further comprises generating count data for the unique aptamer from each of the one or more monoclonal compartments. In some instances, the sequencing further comprises sequences of unique aptamers from the remainder of the monoclonal compartments (e.g., non-bound aptamers). The sequencing further comprises using a sequencer (and optionally an additional assay such as BLI or a spectrometer) to generate sequencing data and optionally analysis data (e.g., a binding-approximation metric and/or functional-approximation metric) for the unique aptamer from each of the remainder of the monoclonal compartments (e.g., non-bound aptamers). The analysis data for the unique aptamer from each of the one or more monoclonal compartments may indicate the unique aptamer did not bind to the one or more targets.

At block 445, the selection sequence data and optionally the count and/or analysis data are used for training a first machine-learning algorithm (e.g., a highly parametric machine-learning algorithm such as a neural network or ensemble of neural networks) to generate a first trained machine-learning model. Thereafter, aptamer sequences are identified, by the first trained machine-learning model, as potentially satisfying one or more constraints, e.g., an initial solution for a given problem. The identification may comprise inputting a subset of sequences from the selection sequence data (from block 440), sequences from a pool of sequences different from the sequences from the selection sequence data, or a combination thereof into the first trained machine-learning model, estimating, by the first trained machine-learning model, a fitness score of each input sequence (the fitness scores is a measure of how well a given sequence performs as a solution with respect to the given problem), and identifying aptamer sequences that satisfy the given problem based on the estimated fitness score for each sequence. In some instances, additional techniques including the application of one or more different types of algorithms such as search algorithms (e.g., a genetic algorithm) or optimization algorithms (e.g., linear optimization) are used in combination with the first trained machine-learning model to improve upon the identification of aptamer sequences. For example, the aptamer sequences identified by the first trained machine-learning model may be evolved using a genetic algorithm to identify or design aptamer sequences that satisfy the given one or more constraints, as described in detail herein.

Optionally at block 450, a count and/or analysis of the identified aptamer sequences is predicted by one or more prediction models. At block 455, the identified aptamer sequences and optionally the predicted analysis data and/or count data are recorded in a data structure in association with the one or more targets.

At block 460, another XNA aptamer library (optionally a DNA or RNA library) is synthesized from the identified aptamer sequences. The aptamers within the another XNA aptamer library (optionally a DNA or RNA library) are partitioned into monoclonal compartments that combined establish another compartment-based capture system. Each monoclonal compartment comprises a unique aptamer from the plurality of aptamers. At block 465, another compartment-based capture system is used to capture the one or more targets. The capturing comprises the one or more targets binding to the unique aptamer sequence within one or more monoclonal compartments. Thereafter, as described similarly with respect to blocks 425-440, the one or more monoclonal compartments of the another compartment-based capture system that comprise the one or more targets bound to the unique aptamer are separated from a remainder of monoclonal compartments of the another compartment-based capture system that does not comprise the one or more targets bound to a unique aptamer. The unique aptamer is then eluted from each of the one or more monoclonal compartments and/or the one or more targets, amplified by enzymatic or chemical processes, and sequenced.

The sequencing comprises using a sequencer (and optionally an additional assay such as BLI or a spectrometer) to generate sequencing data and optionally analysis data (e.g., a binding-approximation metric and/or functional-approximation metric) for the unique aptamer from each of the one or more monoclonal compartments. The analysis data for the unique aptamer from each of the one or more monoclonal compartments may indicate the unique aptamer did bind to the one or more targets. In some instances, the sequencing further comprises generating count data for the unique aptamer from each of the one or more monoclonal compartments. In some instances, the sequencing further comprises sequences of unique aptamers from the remainder of the monoclonal compartments (e.g., non-bound aptamers). The sequencing further comprises using a sequencer (and optionally an additional assay such as BLI or a spectrometer) to generate sequencing data and optionally analysis data (e.g., a binding-approximation metric and/or functional-approximation metric) for the unique aptamer from each of the remainder of the monoclonal compartments (e.g., non-bound aptamers). The analysis data for the unique aptamer from each of the one or more monoclonal compartments may indicate the unique aptamer did not bind to the one or more targets.

Optionally at block 470, the selection sequence data and optionally the count and/or analysis data from block 465 are used as supplemental training data for retraining the first machine-learning algorithm (e.g., a highly parametric machine-learning algorithm such as a neural network or ensemble of neural networks) to generate an improved version of the first trained machine-learning model. The supplemental training data can have a higher accuracy, and/or can have a higher precision relative the accuracy, and/or precision of the training data. For example, the sequences and corresponding count and analysis data in the original training data used in block 445 will have more noise and thus a lower signal to noise ratio, while the noise in the supplemental training data will be lower and thus a higher signal to noise ratio. Thereafter, aptamer sequences are identified, by the improved version of the first trained machine-learning model, as potentially satisfying one or more constraints, e.g., an initial solution for a given problem.

At block 475, some or all of the selection sequence data and optionally the count and/or analysis data (from block 440), the selection sequence data and optionally the count and/or analysis data (from block 465), or a combination thereof are used for training a second machine-learning algorithm (e.g., a linear machine-learning algorithm such as a regression algorithm) to generate a second trained machine-learning model. Thereafter, aptamer sequences are identified, by the second trained machine-learning model, as satisfying the one or more constraints, e.g., a final solution for a given problem. The identification may comprise inputting a subset of sequences from the selection sequence data and optionally the count and/or analysis data (from block 440), a subset of sequences from the selection sequence data and optionally the count and/or analysis data (from block 465), sequences and optionally the count and/or analysis data from a pool of sequences different from the sequences from the selection sequence data, or a combination thereof into the second trained machine-learning model, estimating, by the second trained machine-learning model, a fitness score of each input sequence (the fitness scores is a measure of how well a given sequence performs to potentially satisfy the one or more constraints), and identifying aptamer sequences that potentially satisfy the one or more constraints based on the estimated fitness score for each sequence. In some instances, additional techniques including the application of one or more different types of algorithms such as search algorithms (e.g., a genetic algorithm) or optimization algorithms (e.g., linear optimization) are used in combination with the second trained machine-learning model to improve upon the identification or design of sequences for derived aptamers. For example, identification, by the second trained machine-learning model, of the aptamer sequences may be optimized using an optimization algorithm to identify or design aptamer sequences that potentially satisfy the one or more constraints.

Optionally at block 480, a count or analysis of the identified aptamer sequences is predicted by one or more prediction models. At block 485, the identified aptamer sequences and optionally the predicted analysis data and/or count data are recorded in a data structure in association with the one or more targets

At block 490, the aptamer sequences identified in block 475 may be synthesized and tested for satisfying the one or more constraints (e.g., binding to or inhibiting the target). The testing may include one or more experimental steps comprising BLI and/or functional assays. The BLI and/or functional assays can generate analytical data for the aptamer and target interactions. The interactions may include binding-approximation metrics such as binding and dissociation metrics and/or functional-approximation metrics such as inhibition and promoter metrics.

Optionally at block 495, the selection sequence data and optionally the count and/or analysis data from blocks 475-490 are used as supplemental training data for retraining the first machine-learning algorithm (e.g., a highly parametric machine-learning algorithm such as a neural network or ensemble of neural networks) and/or the second machine-learning algorithm (e.g., a linear machine-learning algorithm such as a regression algorithm) to generate an improved version of the first trained machine-learning model and/or the second trained machine-learning model. The supplemental training data can have a higher accuracy, and/or can have a higher precision relative the accuracy, and/or precision of the training data. For example, the sequences and corresponding count and analysis data in the original training data used in block 445 and/or 465 will have more noise and thus a lower signal to noise ratio, while the noise in the supplemental training data will be lower and thus a higher signal to noise ratio. Moreover, the binding-approximation metric and/or functional-approximation metric in the training data in block 445 and/or 465 may include a binary value (because of use of a high-throughput system), while the binding affinity scores in the supplemental training data may include a categorical or numeric value (because of the BLI and/or functional assays). As another example, the binding-approximation metric and/or functional-approximation metric in the training data can include a categorical value (identifying a category within a first set of categories), while the binding-approximation metric and/or functional-approximation metric in the supplemental training data can include a categorical (identifying a category within a second set of categories, where there are more categories in the second set relative to the first set) or numeric value. As yet another example, the binding-approximation metric and/or functional-approximation metric in the training data can include a numeric value with a first number of significant figures, while the binding-approximation metric and/or functional-approximation metric in the supplemental training data can include a numeric value with more significant figurers than the first number of significant figures. Thereafter, aptamer sequences are identified, by the improved version of the first trained machine-learning model and/or second trained machine-learning model, as potentially satisfying the one or more constraints, e.g., an initial solution for a given problem.

At block 497, the analytical data generated in 490 may be used to generate or curate a final set of aptamer sequences as satisfying the one or more constraints, e.g., a final solution to a given problem. In some instances, an output library is generated that comprises the final set of aptamer sequences. The output library may be generated incrementally as new aptamer sequences are generated and selected by repeating all or select blocks of blocks 405-495 (e.g., several cycles of 400 or a portion thereof in a loop or interlaced loops). At each repetition cycle one or more aptamer sequences may be identified (i.e., designed, generated and/or selected) and added to the output based on their ability to satisfy the one or more constraints.

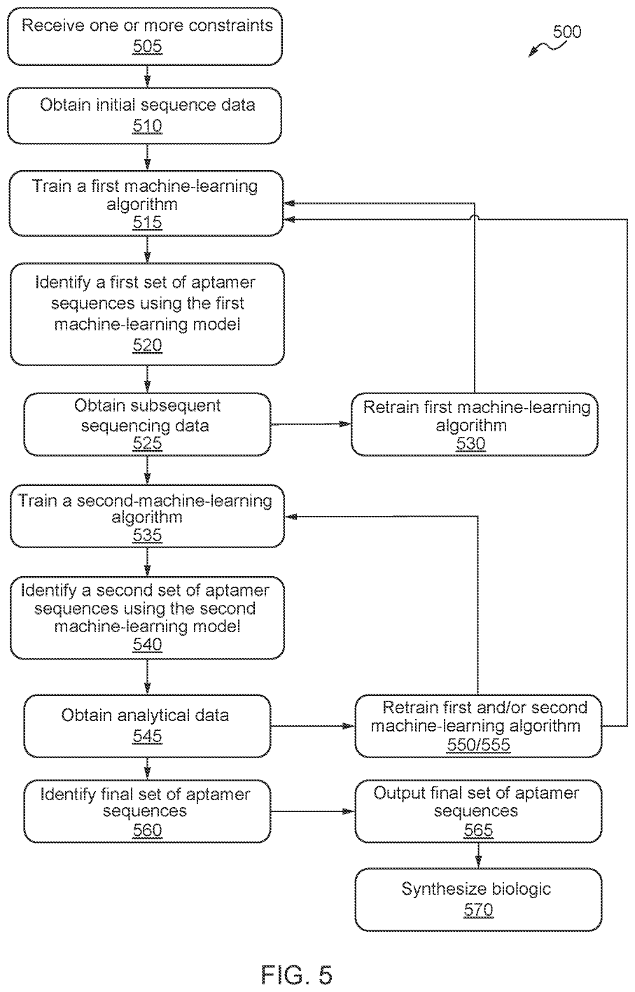

FIG. 5 is a simplified flowchart 500 illustrating an example of the process for developing aptamers using a machine-learning modeling system and an aptamer development platform (e.g., machine-learning modeling system 200 and the aptamer development platform 300 described with respect to FIGS. 2 and 3). Process 500 begins at block 505, at which one or more constraints are received concerning potential therapeutic candidates that can bind a target. For example, a user may pose a query (or a given problem) concerning identification of a hundred aptamers with the highest binding affinity for a given target or a hundred aptamers with the greatest ability to inhibit activity of a given target. At block 510, initial sequence data is obtained for aptamers of an initial aptamer library that bind to a target, do not bind to the target, or a combination thereof. The initial sequence data may be obtained using an in vitro binding selection process (e.g., SELEX), as described in detail with respect to flowchart 400 depicted in FIGS. 4A and 4B. The initial sequence data comprises aptamer sequences and, optionally, associated analytical data. The analytical data comprising a first binding-approximation metric, a first functional-approximation metric, or a combination thereof of the aptamers derived from the aptamer sequences.

At block 515, a first machine-learning algorithm is trained using the initial sequence data to learn model parameters and generate the first machine-learning model. At block 520, a first set of aptamer sequences is identified, by the first machine-learning model, as satisfying the one or more constraints. The first set of aptamer sequences are derived from a subset of sequences from the initial sequence data, sequences from a pool of sequences different from sequences from the initial sequence data, or a combination thereof. At block 525, subsequent sequence data is obtained, using an in vitro binding selection process (e.g., SELEX), for aptamers of a subsequent aptamer library that bind to the target, do not bind to the target, or a combination thereof. The subsequent aptamer library comprises aptamers synthesized from the first set of aptamer sequences. The subsequent sequence data comprises aptamer sequences and, optionally, associated analytical data. The analytical data comprising a second binding-approximation metric, a second functional-approximation metric, or a combination thereof of aptamers derived from the aptamer sequences

At block 530, the first machine-learning algorithm may be retrained using the subsequent sequence data to relearn the model parameters and generate another version of the first machine-learning model. Thereafter, block 520 may be repeated using the another version of the first machine-learning model to identify a revised first set of aptamer sequences as satisfying the one or more constraints; and block 525 may be repeated to identify subsequent sequence data, where the subsequent aptamer library comprises aptamers synthesized from the revised first set of aptamer sequences.

At block 535, a second machine-learning algorithm is trained using the subsequent sequence data to learn model parameters and generate a second machine-learning model. At block 540, a second set of aptamer sequences is identified, by the second machine-learning model, as satisfying the one or more constraints. The second set of aptamer sequences are derived from a subset of sequences from the subsequent sequence data, sequences from a pool of sequences different from sequences from the subsequent sequence data, or a combination thereof. At block 545, analytical data for aptamers synthesized from the second set of aptamer sequences is obtained using one or more in vitro assays (e.g., BLI). The analytical data comprises a third binding-approximation metric, a third functional-approximation metric, or a combination thereof of aptamers derived from the second set of aptamer sequences.

At block 550, the first machine-learning algorithm may be retrained using the second set of aptamer sequences and the analytical data for aptamers derived from the second set of aptamer sequences to relearn the model parameters and generate another version of the first machine-learning model. Thereafter, block 520 may be repeated using the another version of the first machine-learning model to identify a revised first set of aptamer sequences as satisfying the one or more constraints; and block 525 may be repeated to identify revised subsequent sequence data, where the subsequent aptamer library comprises aptamers synthesized from the revised first set of aptamer sequences. Thereafter, blocks 540 and 545 may be repeated based on the revised subsequent sequence data.

At block 555, the second machine-learning algorithm may be retrained using the second set of aptamer sequences and the analytical data for aptamers derived from the second set of aptamer sequences to relearn the model parameters and generate another version of the second machine-learning model. Thereafter, block 540 may be repeated using the another version of the second machine-learning model to identify a revised second set of aptamer sequences as satisfying the one or more constraints; and block 545 may be repeated to determine analytical data for aptamers derived from the revised second set of aptamer sequences.

At block 560, a final set of aptamer sequences is identified from the second set of aptamer sequences that satisfy the one or more constraints based on the analytical data associated with each aptamer. For example the analytical data for each aptamer may be compared to the one or more constraints, and a determination made as to whether the aptamer satisfies the one or more constraints based on the comparison. At block 565, the final set of aptamer sequences is output. In some instances, the output comprises the final set of aptamer sequences and an identifier of a target (e.g., name of a protein or an identifier from a database such as the protein databank identifier) corresponding to each of the aptamers in the final set. In some instances, the final set of aptamer sequences is locally presented (e.g., displayed) or transmitted to another device. In some instances, the final set of aptamer sequences is output to an end user or storage device. In some instances, the final set of aptamer sequences is output to an end user or storage device as a final solution to a given problem (or a result to the query).

At optional block 570, a biologic is synthesized using the one or more aptamers validated as satisfying the one or more constraints. The biologic may be used as a new drug, a therapeutic tool, a drug delivery device, diagnosis of disease, bio-imaging, analytical reagent, hazard detection, food inspection, and the like. At optional block 565, a treatment is administered to a subject with the biologic.

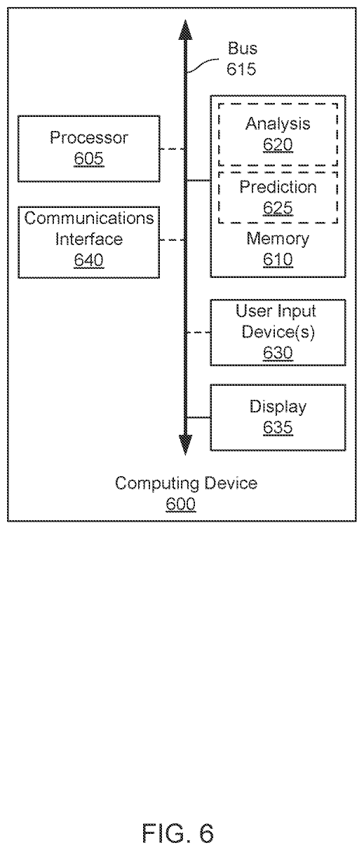

FIG. 6 illustrates an example computing device 600 suitable for use with systems and methods for developing aptamers and biologics or providing results to a query according to this disclosure. The example computing device 600 includes a processor 605 which is in communication with the memory 610 and other components of the computing device 600 using one or more communications buses 615. The processor 605 is configured to execute processor-executable instructions stored in the memory 610 to perform one or more methods for developing aptamers or biologics or providing results to a query according to different examples, such as part or all of the example method 400 or 500 described above with respect to FIGS. 4 and 5. In this example, the memory 610 stores processor-executable instructions that provide for provisioning of machine-learning algorithms or models 620 and aptamer identification 625, as discussed above with respect to FIGS. 1-5 (e.g., the controller/computer of platform 300).

The computing device 600, in this example, also includes one or more user input devices 630, such as a keyboard, mouse, touchscreen, microphone, etc., to accept user input. The computing device 600 also includes a display 635 to provide visual output to a user such as a user interface or display of aptamer sequences. The computing device 600 also includes a communications interface 640. In some examples, the communications interface 640 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### V. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A method comprising:
(a) obtaining initial sequence data for aptamers of an initial aptamer library that bind to a target, do not bind to the target, or a combination thereof;
(b) identifying, by a first machine-learning model, a first set of aptamer sequences as satisfying one or more constraints, wherein the first machine-learning model comprises model parameters learned from the initial sequence data, and the first set of aptamer sequences are derived from a subset of sequences from the initial sequence data, sequences from a pool of sequences different from sequences from the initial sequence data, or a combination thereof;
(c) obtaining, using an in vitro binding selection process, subsequent sequence data for aptamers of a subsequent aptamer library that bind to the target, do not bind to the target, or a combination thereof, wherein the subsequent aptamer library comprises aptamers synthesized from the first set of aptamer sequences;
(d) identifying, by a second machine-learning model, a second set of aptamer sequences as satisfying the one or more constraints, wherein the second machine-learning model comprises model parameters learned from the subsequent sequence data, and the second set of aptamer sequences are derived from a subset of sequences from the subsequent sequence data, sequences from a pool of sequences different from sequences from the subsequent sequence data, or a combination thereof;
(e) determining, using one or more in vitro assays, analytical data for aptamers synthesized from the second set of aptamer sequences;
(f) identifying a final set of aptamer sequences from the second set of aptamer sequences that satisfy the one or more constraints based on the analytical data associated with each aptamer; and
(g) outputting the final set of aptamer sequences.

2. The method of claim 1, further comprising training the first machine-learning algorithm using the initial sequence data to learn the model parameters and generate the first machine-learning model, wherein the initial sequence data comprises aptamer sequences and associated analytical data, the analytical data comprising a first binding-approximation metric, a first functional-approximation metric, or a combination thereof of aptamers derived from the aptamer sequences.

3. The method of claim 2, further comprising training the second machine-learning algorithm using the subsequent sequence data to learn the model parameters and generate the second machine-learning model, wherein the subsequent sequence data comprises aptamer sequences and associated analytical data, the analytical data comprising a second binding-approximation metric, a second functional-approximation metric, or a combination thereof of aptamers derived from the aptamer sequences.

4. The method of claim 2, further comprising:
prior to identifying the second set of aptamer sequences, retraining the first machine-learning algorithm using the subsequent sequence data to relearn the model parameters and generate another version of the first machine-learning model, wherein the subsequent sequence data comprises aptamer sequences and associated analytical data, the analytical data comprising a second binding-approximation metric, a second functional-approximation metric, or a combination thereof of aptamers derived from the aptamer sequences;
repeating step (b) using the another version of the first machine-learning model to identify a revised first set of aptamer sequences as satisfying the one or more constraints; and
repeating step (c) to identify subsequent sequence data wherein the subsequent aptamer library comprises aptamers synthesized from the revised first set of aptamer sequences.

5. The method of claim 3, further comprising:
prior to identifying the final set of aptamers, retraining the first machine-learning algorithm using the second set of aptamer sequences and the analytical data for aptamers derived from the second set of aptamer sequences to relearn the model parameters and generate another version of the first machine-learning model, wherein the analytical data comprises a third binding-approximation metric, a third functional-approximation metric, or a combination thereof of aptamers derived from the second set of aptamer sequences;
repeating step (b) using the another version of the first machine-learning model to identify a revised first set of aptamer sequences as satisfying the one or more constraints;
repeating step (c) to obtain revised subsequent sequence data, wherein the subsequent aptamer library comprises aptamers synthesized from the revised first set of aptamer sequences; and
repeating steps (d)-(e) based on the revised subsequent sequence data.

6. The method of claim 3, further comprising:
prior to identifying the final set of aptamers, retraining the second machine-learning algorithm using the second set of aptamer sequences and the analytical data for aptamers derived from the second set of aptamer sequences to relearn the model parameters and generate another version of the second machine-learning model, wherein the analytical data comprises a third binding-approximation metric, a third functional-approximation metric, or a combination thereof of aptamers derived from the second set of aptamer sequences;
repeating step (d) using the another version of the second machine-learning model to identify a revised second set of aptamer sequences as satisfying the one or more constraints; and
repeating step (e) to determine analytical data for aptamers derived from the revised second set of aptamer sequences.

7. The method of claim 1, further comprising:
synthesizing one or more aptamers using the final set of aptamer sequences; and
synthesizing a biologic using the one or more aptamers.

8. A computer-program product tangibly embodied in a non-transitory machine-readable storage medium, including instructions configured to cause one or more data processors to perform actions including:
(a) obtaining initial sequence data for aptamers of an initial aptamer library that bind to a target, do not bind to the target, or a combination thereof;
(b) identifying, by a first machine-learning model, a first set of aptamer sequences as satisfying one or more constraints, wherein the first machine-learning model comprises model parameters learned from the initial sequence data, and the first set of aptamer sequences are derived from a subset of sequences from the initial sequence data, sequences from a pool of sequences different from sequences from the initial sequence data, or a combination thereof;
(c) obtaining, using an in vitro binding selection process, subsequent sequence data for aptamers of a subsequent aptamer library that bind to the target, do not bind to the target, or a combination thereof, wherein the subsequent aptamer library comprises aptamers synthesized from the first set of aptamer sequences;
(d) identifying, by a second machine-learning model, a second set of aptamer sequences as satisfying the one or more constraints, wherein the second machine-learning model comprises model parameters learned from the subsequent sequence data, and the second set of aptamer sequences are derived from a subset of sequences from the subsequent sequence data, sequences from a pool of sequences different from sequences from the subsequent sequence data, or a combination thereof;
(e) determining, using one or more in vitro assays, analytical data for aptamers synthesized from the second set of aptamer sequences;
(f) identifying a final set of aptamer sequences from the second set of aptamer sequences that satisfy the one or more constraints based on the analytical data associated with each aptamer; and
(g) outputting the final set of aptamer sequences.

9. The computer-program product of claim 8, wherein the actions further comprise training the first machine-learning algorithm using the initial sequence data to learn the model parameters and generate the first machine-learning model, wherein the initial sequence data comprises aptamer sequences and associated analytical data, the analytical data comprising a first binding-approximation metric, a first functional-approximation metric, or a combination thereof of aptamers derived from the aptamer sequences.

10. The computer-program product of claim 9, wherein the actions further comprise training the second machine-learning algorithm using the subsequent sequence data to learn the model parameters and generate the second machine-learning model, wherein the subsequent sequence data comprises aptamer sequences and associated analytical data, the analytical data comprising a second binding-approximation metric, a second functional-approximation metric, or a combination thereof of aptamers derived from the aptamer sequences.

11. The computer-program product of claim 9, wherein the actions further comprise:
prior to identifying the second set of aptamer sequences, retraining the first machine-learning algorithm using the subsequent sequence data to relearn the model parameters and generate another version of the first machine-learning model, wherein the subsequent sequence data comprises aptamer sequences and associated analytical data, the analytical data comprising a second binding-approximation metric, a second functional-approximation metric, or a combination thereof of aptamers derived from the aptamer sequences;
repeating step (b) using the another version of the first machine-learning model to identify a revised first set of aptamer sequences as satisfying the one or more constraints; and
repeating step (c) to identify subsequent sequence data wherein the subsequent aptamer library comprises aptamers synthesized from the revised first set of aptamer sequences.

12. The computer-program product of claim 10, wherein the actions further comprise:
prior to identifying the final set of aptamers, retraining the first machine-learning algorithm using the second set of aptamer sequences and the analytical data for aptamers derived from the second set of aptamer sequences to relearn the model parameters and generate another version of the first machine-learning model, wherein the analytical data comprises a third binding-approximation metric, a third functional-approximation metric, or a combination thereof of aptamers derived from the second set of aptamer sequences;
repeating step (b) using the another version of the first machine-learning model to identify a revised first set of aptamer sequences as satisfying the one or more constraints;
repeating step (c) to obtain revised subsequent sequence data, wherein the subsequent aptamer library comprises aptamers synthesized from the revised first set of aptamer sequences; and
repeating steps (d)-(e) based on the revised subsequent sequence data.

13. The computer-program product of claim 10, wherein the actions further comprise:
prior to identifying the final set of aptamers, retraining the second machine-learning algorithm using the second set of aptamer sequences and the analytical data for aptamers derived from the second set of aptamer sequences to relearn the model parameters and generate another version of the second machine-learning model, wherein the analytical data comprises a third binding-approximation metric, a third functional-approximation metric, or a combination thereof of aptamers derived from the second set of aptamer sequences;
repeating step (d) using the another version of the second machine-learning model to identify a revised second set of aptamer sequences as satisfying the one or more constraints; and
repeating step (e) to determine analytical data for aptamers derived from the revised second set of aptamer sequences.

14. The computer-program product of claim 1, wherein the actions further comprise:
synthesizing one or more aptamers using the final set of aptamer sequences; and
synthesizing a biologic using the one or more aptamers.

15. A system comprising:
one or more data processors; and
a non-transitory computer readable storage medium containing instructions which, when executed on the one or more data processors, cause the one or more data processors to perform actions including:
(a) obtaining initial sequence data for aptamers of an initial aptamer library that bind to a target, do not bind to the target, or a combination thereof;
(b) identifying, by a first machine-learning model, a first set of aptamer sequences as satisfying one or more constraints, wherein the first machine-learning model comprises model parameters learned from the initial sequence data, and the first set of aptamer sequences are derived from a subset of sequences from the initial sequence data, sequences from a pool of sequences different from sequences from the initial sequence data, or a combination thereof;
(c) obtaining, using an in vitro binding selection process, subsequent sequence data for aptamers of a subsequent aptamer library that bind to the target, do not bind to the target, or a combination thereof, wherein the subsequent aptamer library comprises aptamers synthesized from the first set of aptamer sequences;
(d) identifying, by a second machine-learning model, a second set of aptamer sequences as satisfying the one or more constraints, wherein the second machine-learning model comprises model parameters learned from the subsequent sequence data, and the second set of aptamer sequences are derived from a subset of sequences from the subsequent sequence data, sequences from a pool of sequences different from sequences from the subsequent sequence data, or a combination thereof;
(e) determining, using one or more in vitro assays, analytical data for aptamers synthesized from the second set of aptamer sequences;
(f) identifying a final set of aptamer sequences from the second set of aptamer sequences that satisfy the one or more constraints based on the analytical data associated with each aptamer; and
(g) outputting the final set of aptamer sequences.

16. The system of claim 15, wherein the actions further comprise training the first machine-learning algorithm using the initial sequence data to learn the model parameters and generate the first machine-learning model, wherein the initial sequence data comprises aptamer sequences and associated analytical data, the analytical data comprising a first binding-approximation metric, a first functional-approximation metric, or a combination thereof of aptamers derived from the aptamer sequences.

17. The system of claim 16, wherein the actions further comprise training the second machine-learning algorithm using the subsequent sequence data to learn the model parameters and generate the second machine-learning model, wherein the subsequent sequence data comprises aptamer sequences and associated analytical data, the analytical data comprising a second binding-approximation metric, a second functional-approximation metric, or a combination thereof of aptamers derived from the aptamer sequences.

18. The system of claim 16, wherein the actions further comprise:
prior to identifying the second set of aptamer sequences, retraining the first machine-learning algorithm using the subsequent sequence data to relearn the model parameters and generate another version of the first machine-learning model, wherein the subsequent sequence data comprises aptamer sequences and associated analytical data, the analytical data comprising a second binding-approximation metric, a second functional-approximation metric, or a combination thereof of aptamers derived from the aptamer sequences;
repeating step (b) using the another version of the first machine-learning model to identify a revised first set of aptamer sequences as satisfying the one or more constraints; and
repeating step (c) to identify subsequent sequence data wherein the subsequent aptamer library comprises aptamers synthesized from the revised first set of aptamer sequences.

19. The system of claim 17, wherein the actions further comprise:
prior to identifying the final set of aptamers, retraining the first machine-learning algorithm using the second set of aptamer sequences and the analytical data for aptamers derived from the second set of aptamer sequences to relearn the model parameters and generate another version of the first machine-learning model, wherein the analytical data comprises a third binding-approximation metric, a third functional-approximation metric, or a combination thereof of aptamers derived from the second set of aptamer sequences;
repeating step (b) using the another version of the first machine-learning model to identify a revised first set of aptamer sequences as satisfying the one or more constraints;
repeating step (c) to obtain revised subsequent sequence data, wherein the subsequent aptamer library comprises aptamers synthesized from the revised first set of aptamer sequences; and
repeating steps (d)-(e) based on the revised subsequent sequence data.

20. The system of claim 17, wherein the actions further comprise:
prior to identifying the final set of aptamers, retraining the second machine-learning algorithm using the second set of aptamer sequences and the analytical data for aptamers derived from the second set of aptamer sequences to relearn the model parameters and generate another version of the second machine-learning model, wherein the analytical data comprises a third binding-approximation metric, a third functional-approximation metric, or a combination thereof of aptamers derived from the second set of aptamer sequences;
repeating step (d) using the another version of the second machine-learning model to identify a revised second set of aptamer sequences as satisfying the one or more constraints; and
repeating step (e) to determine analytical data for aptamers derived from the revised second set of aptamer sequences.
